(19) United States
(12) Patent Application Publication
(10) Pub. No.: US 2026/0290520 A1
(43) Pub. Date: Sep. 24, 2026

(54) RETROSYNTHESIS PREDICTION SYSTEM AND METHOD USING GRAPH GENERATIVE MODELS

(71) Applicant: Ohio State Innovation Foundation, Columbus, OH (US)

(72) Inventors: Xia NING, Columbus, OH (US); Ziqi CHEN, Columbus, OH (US)

(21) Appl. No.: 18/871,585

(22) PCT Filed: Apr. 5, 2023
(86) PCT No.: PCT/US2023/017546

§ 371 Date: Dec. 4, 2024

Related U.S. Application Data
(60) Provisional application No. 63/349,129, filed on Jun. 5, 2022.

(51) Int. Cl. G16C 20/50; G06F 30/27; G16C 20/10; G16C 20/70; G16C 20/80

(52) U.S. Cl. CPC G16C 20/50; G06F 30/27; G16C 20/10; G16C 20/70; G16C 20/80

(57) ABSTRACT

An exemplary retrosynthesis prediction method and system, e.g., for a semi-template-based approach, are disclosed that imitates the reversed logic of synthetic reactions: first predicting the reaction centers in target molecules, identifies the synthons needed to assemble the final products, and the transforms these synthons into reactants. Among other things, the exemplary retrosynthesis prediction method and system can learn from molecular graphs that encode information most directly relevant to synthetic transformations—considering not only the structures of the synthons to be completed but also the structures of other synthons from the same products so as to utilize their relations during reactions.

### RELATED APPLICATION

This PCT application claims priority to, and the benefit of, U.S. Provisional Patent Application No. 63/349,129, filed Jun. 5, 2022, entitled “Retrosynthesis Prediction System and Method using Graph Generative Models,” which is hereby incorporated by reference herein in its entirety.

### STATEMENT OF GOVERNMENT SUPPORT

This invention was made with government support under Grant No. IIS-2133650 awarded by the National Science Foundation. The government has certain rights in the invention.

### BACKGROUND

A time-consuming and costly step in drug development is the identification of drug-like small molecules that display desired properties against a specific biomolecular target and then the synthesis of such molecules if they do not exist. Retrosynthesis is a procedure where such a desired molecule is transformed into potential reactants, and thus, the synthesis routes are identified. The success and efficiency of retrosynthesis of drug-like small molecules immensely impact the entire drug development process and affect its success rate, costs, and speed.

Current retrosynthesis analyses are primarily conducted by synthetic and medicinal chemists based on their knowledge and experience, which could be limited or susceptible to human error. Consequently, the planned synthetic routes may not be diverse enough to cover novel, economical, or green reactions.

Recent in silico retrosynthesis prediction methods (i.e., simulations) using deep learning have enabled alternative computationally generative processes to accelerate the conventional paradigm. These deep-learning methods learn from string-based representations (SMILES) or graph representations of given molecules and can generate possible reactant structures that can be used to synthesize target molecules, leveraging the advancement of natural language processing, graph neural networks, auto-encoders, and other techniques in deep learning.

Nevertheless, they may consider only a limited number of structures of the synthons to be completed, a limited set of reaction centers, a limited set of substructures for attachment when completing synthons into reactants, or a limited set of representation of the molecular product. In addition, current in silico retrosynthesis prediction methods may generate too many results that may not be sufficiently diverse from one another, thus providing a limited search of potential candidates.

There is thus a strong need and benefit to improving retrosynthesis predictions of reactants that can be used in the synthesis of a target molecule.

### SUMMARY

An exemplary retrosynthesis prediction method and system, e.g., for a semi-template-based approach, are disclosed that imitates the reversed logic of synthetic reactions: first predicting the reaction centers in target molecules (denoted herein by “products”), identifies the fragments (denoted herein as “synthons”) needed to assemble the final products, and then transforms these synthons into reactants. The operation facilitates step-wise interpretability. The exemplary retrosynthesis prediction method and system can learn from molecular graphs that encode information most directly relevant to synthetic transformations—considering not only the structures of the synthons to be completed but also the structures of other synthons from the same products so as to utilize their relations during reactions.

The exemplary retrosynthesis prediction method and system can employ trained deep graph neural networks optimally configured to operate over molecular graphs that can better capture the synthetic reactions of molecular structures. The trained deep graph neural networks can be used to predict each type of the reaction centers with high accuracies, e.g., by considering multiple reaction center candidates for each product.

The trained deep graph neural networks can learn from a plurality of molecular graphs of molecular products and encodes their molecule structures most indicative of multiple reaction center types. The trained deep graph neural networks can operate over different molecular graph types, e.g., graphs having fragment information, and using them in combination with molecular graphs in molecule structure learning.

The exemplary retrosynthesis prediction method and system, when completing synthons, may employ a holistic view of all involved synthon and product structures, as well as their complementary relations, to identify an optimal completion action. The exemplary retrosynthesis prediction method and system, via trained deep graph neural networks, can evaluate a comprehensive set of reaction center types (e.g., covering 97.5%⁺ of a given test data comprising a 50K⁺ database) and conform to synthetic chemistry knowledge.

In addition, the exemplary retrosynthesis prediction method and system can complete synthons by sequentially adding very small substructures (e.g., bonds and/or rings) in addition to single atoms or bonds.

To limit the exhaustive, full-scale generation of all possible reactions, thus facilitating scalability, the exemplary retrosynthesis prediction method and system may be configured to prioritize the most possible reactions using a beam search strategy that optimally prioritizes the most possible reactants along the synthon completion paths.

In an aspect, a method is disclosed to perform retrosynthesis to predict reactants that can be used in a synthetic molecule model, the method comprising: receiving, by a processor, the input molecule data structure represented as a graph data object (e.g., a molecular graph model, a BRICS (associated with fragments determined by Breaking-Retrosynthetically-Interesting-Chemical substructures fragmentation algorithm) graph model, or a junction tree graph model); evaluating, by the processor, the one or more graph data objects of the input molecule data structure, wherein includes a set of sequences to: determine, by the processor, via one or more a trained AI algorithm (e.g., neural network operations) applied via a training data set of molecule data structures, for one or more reaction center types, a likelihood value that a given bond of a plurality of assessed bonds or a given atom of a plurality of assessed atoms of each of one or more graph data objects is likely a reaction center for a subsequent synthesis (e.g., determining S^{b}, S^{c}, and/or S^{a} as described herein), wherein the one or more reaction center types include: i) a first reaction center type associated with a new chemical bond formation that is formed during a hypothetical reaction and exists in the input target molecules but not in the output reactants molecules (i.e., BF-center); ii) a second reaction center type associated with an existing chemical bond in the input molecule data structure being changed in type during a hypothetical reaction (e.g., due to a gain or a loss of hydrogens; i.e., BC-center); and/or iii) a third reaction center type associated with an atom in the input molecule data structure connected with fragments being removed during a hypothetical reaction without a new chemical bond being formed or a type of the existing chemical bond being changed (i.e., A-center), wherein the likelihood values of the plurality of assessed bonds of each of one or more graph data objects are used in a selection of one or more reaction centers for the one or more graph data objects to generate a plurality of intermediate molecular graph data objects (e.g., synthon graphs; S^{b} from BF-center, S^{c} from BC-center, S^{a} for A-center) each having a modified chemical bond at the selected one or more reaction centers for the one or more graph data objects, wherein one or more of the plurality of intermediate molecular graph data objects with the modified chemical bond are attached to a chemical fragment at the modified chemical bond to generate a completed reactant graph, wherein the completed reactant graph is used to derive the synthetic molecule model.

In some embodiments, the one or more of the plurality of intermediate molecular graph data objects with the modified chemical bond are generated by determining, by the processor, a likelihood of a neighbor bond type change for a given reaction center.

In some embodiments, the likelihood of the neighbor bond type change for the given reaction center is determined using a second trained AI algorithm (e.g., second neural network, e.g., comprising a SoftMax operator) that learns for a given bond structure and on a structure of the graph data objects (e.g., hₚ and bᵢⱼ) of the training data set of molecule data structures (e.g., via Equation 13).

In some embodiments, the one or more of the plurality of intermediate molecular graph data objects with the modified chemical bond are generated by determining, by the processor, a likelihood of an atom charge change for all the atoms involved in a given reaction center and their associated neighboring bonds (e.g., for BF-center).

In some embodiments, the likelihood of the atom charge change is determined using a third AI algorithm (e.g., third neural network, e.g., comprising a SoftMax operator) that learns via training data set of molecule data structures (e.g., via Equation 16), for a given bond structure probabilities of accepting one electron, donating one electron, or no electron change during a hypothetical reaction

In some embodiments, the one or more of the plurality of intermediate molecular graph data objects with the modified chemical bond are generated by iteratively attaching, by the processor, a chemical substructure to a given intermediate molecular graph data object of the plurality of intermediate molecular graph data objects based on a fourth train AI algorithm (e.g., fourth neural network, e.g., comprising a sigmoid function, e.g., Equation 18) that learns for a given atom structure and on a structure of the graph data objects (e.g., hₚ and hₛ) of the training data set of molecule data structures.

In some embodiments, the step of iteratively attaching the chemical substructure includes determining, by the processor, the likelihood values for inclusion of one or more types of new substructure from a library of candidate substructures (e.g., set of vocabulary {z}) each comprising a bond or a ring structure, wherein the likelihood values are determined using a fifth trained AI algorithm (e.g., fifth neural network, e.g., comprising a SoftMax operator, e.g., Equation 20) that learns for a given atom structure and on a structure of the graph data objects (e.g., hₚ and hₛ) of the training data set of molecule data structures.

In some embodiments, the one or more graph data objects include a molecular graph and a BRICS graph (e.g., associated with a Breaking-Retrosynthetically-Interesting-Chemical substructures fragmentation algorithm).

In some embodiments, the one or more graph data objects include a molecular graph and a junction tree.

In some embodiments, the selection of one or more reactions for the one or more graph data objects includes generating, by the processor, a ranked list of candidate reactant graphs; and selecting, by the processor, via a beam search, a pre-defined number of top reaction centers as the one or more reaction centers for the one or more graph data objects to generate the plurality of intermediate molecular graph data objects.

In some embodiments, the likelihood value that the given bond of the plurality of assessed bonds of each of one or more graph data objects is likely the first reaction center type (BF-center) is determined based on a sixth trained AI algorithm (e.g., sixth neural network), wherein the sixth trained AI algorithm learn (e.g., using Equation 8), using the training data set of molecule data structures, based on a given bond structure and on a structure of the graph data objects (e.g., hₚ and bᵢⱼ).

In some embodiments, the likelihood value that the given bond of the plurality of assessed bonds of each of one or more graph data objects is likely the second reaction center type (BC-center) is determined based on a seventh trained AI algorithm (e.g., seventh neural network), wherein the seventh trained AI algorithm learns (e.g., using Equation 9), using the training data set of molecule data structures, based on a given bond structure and on a structure of the graph data objects (e.g., hₚ and bᵢⱼ).

In some embodiments, the likelihood value that the given atom of the plurality of assessed atoms of each of one or more graph data objects is likely the third reaction center type (A-center) is determined based on an eighth trained AI algorithm (e.g., eighth neural network), wherein the eighth trained AI algorithm learns (using Equation 10), using the training data set of molecule data structures, based on an atom and a structure of the graph data objects (e.g., aᵢ and hₚ).

In some embodiments, the input molecule data structure is evaluated along with a plurality of input molecule data structures in a pipeline operation.

In some embodiments, the plurality of input molecule data structures has an average molecule size between 20-500 atoms or rings.

In some embodiments, the input molecule data structure is of an existing therapeutic molecule or a pharmaceutically active molecule.

In some embodiments, the chemical substructure is iteratively attached in a ranked manner from the library of candidate substructures without any chemical rules or templates imposed for the attachment (e.g., in a template-free manner).

In some embodiments, wherein the input molecule data structure was generated by molecule optimization evaluation for drug discovery analysis.

In another aspect, a system is disclosed comprising a processor; and a memory operatively connected to the processor, the memory having instructions stored thereon, wherein execution by the processor causes the processor to perform any of the above-discussed methods.

In another aspect, a non-transitory computer-readable medium is disclosed having instructions stored thereon, wherein execution by the processor causes the processor to perform any of the methods of the above-discussed methods.

### BRIEF DESCRIPTION OF THE DRAWINGS

The skilled person in the art will understand that the drawings described below are for illustration purposes only.

FIG. 1 shows an example retrosynthesis prediction system configured to generate candidate reactants for a given target molecule in accordance with an illustrative embodiment.

FIG. 2A shows an example operation of the retrosynthesis prediction system of FIG. 1 in accordance with an illustrative embodiment.

FIG. 2B shows an example pipeline operation that integrated the retrosynthesis operation with a molecular prediction in accordance with an illustrative embodiment.

FIGS. 3A through 3G each shows example outputs of a retrosynthesis prediction system employed in a study in accordance with an illustrative embodiment.

FIG. 3A through 3G present examples of the diverse reactions predicted by the retrosynthesis prediction system of FIG. 1 in accordance with an illustrative embodiment.

FIGS. 4A, 4B, 4C, and 4D show experimental results showing performance of various aspects of the retrosynthesis prediction system in accordance with an illustrative embodiment.

FIG. 5 shows an example set of the substructures that may be used by the exemplary completion module to complete synthons of the retrosynthesis prediction system of FIG. 1 in accordance with an illustrative embodiment.

### DETAILED SPECIFICATION

Some references, which may include various patents, patent applications, and publications, are cited in a reference list and discussed in the disclosure provided herein. The citation and/or discussion of such references is provided merely to clarify the description of the present disclosure and is not an admission that any such reference is “prior art” to any aspects of the present disclosure described herein. In terms of notation, “[n]” corresponds to the n^{th} reference in the list. All references cited and discussed in this specification are incorporated herein by reference in their entireties and to the same extent as if each reference was individually incorporated by reference.

### Example Retrosynthesis Prediction System

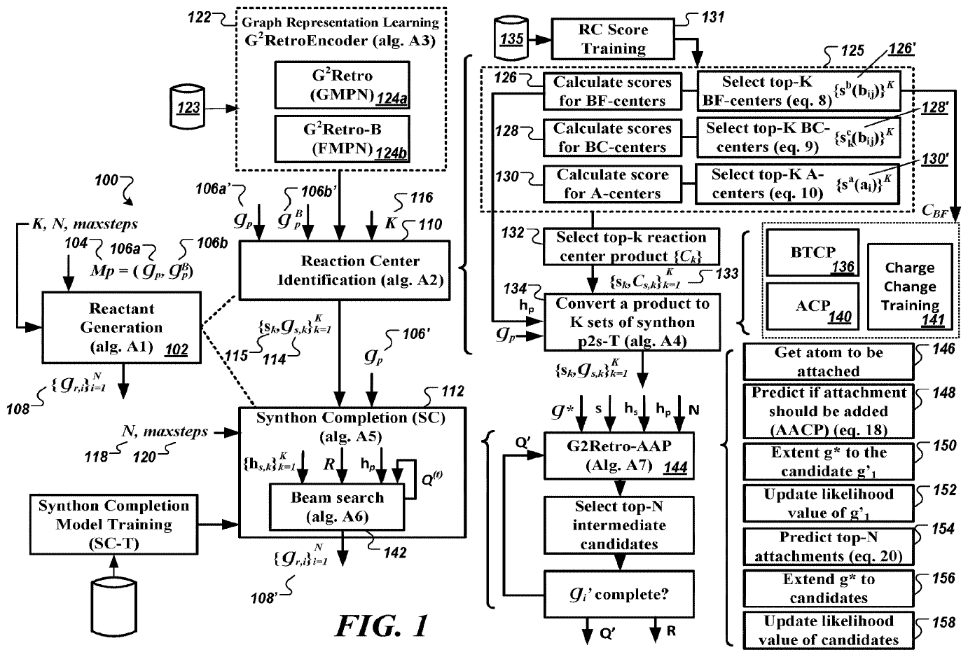

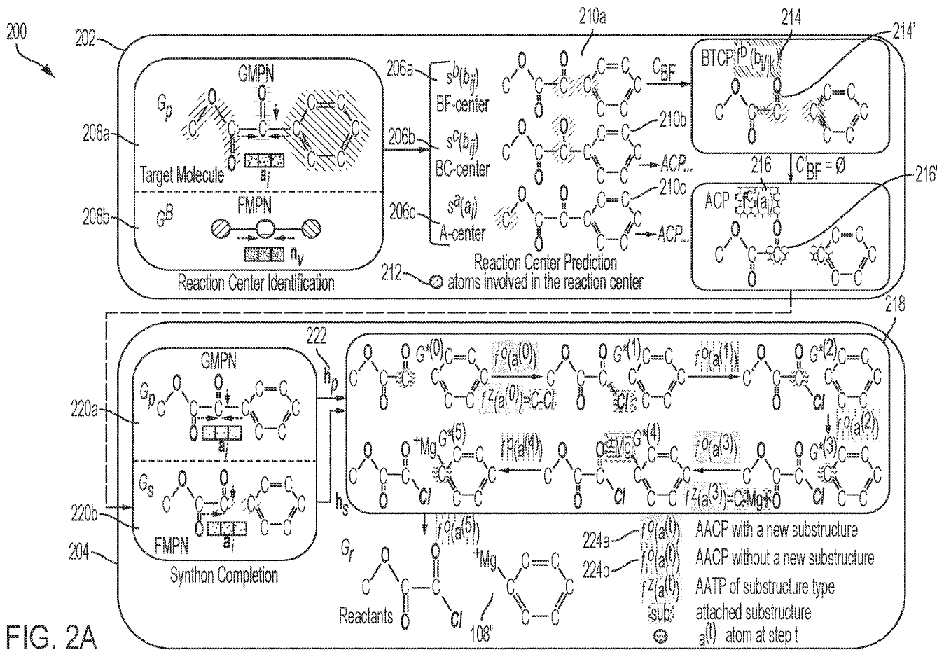

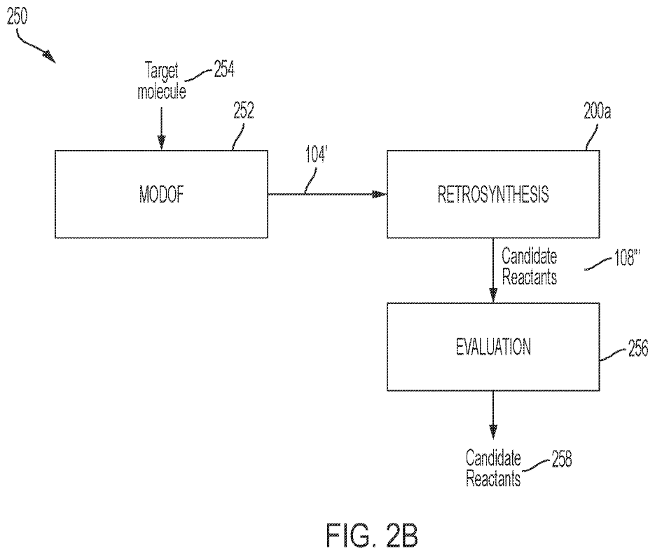

FIG. 1 shows an example retrosynthesis prediction system 100 configured to generate candidate reactants for a given target molecule in accordance with an illustrative embodiment. FIG. 2A shows an example operation 200 of the retrosynthesis prediction system 100 of FIG. 1 in accordance with an illustrative embodiment. The target molecule is likely to have a molecular weight of less than 1,000 Da. The prediction system can be used to evaluate simple compounds, e.g., having 30 atoms or complex compounds having 300-500 atoms.

In the example shown in FIG. 1, the retrosynthesis prediction system 100 includes a reactant generation module 102 configured to receive the target molecule 104 (shown as product molecule “Mₚ” 104) that can be represented in the form of, i.e., generated into, graph generative models 106 (shown as product graph “ [symbol] ” 106a and BRICS graph

$$“ {𝒢}_{p}^{B} ”$$

106b) to generate a set (N number of candidates) of candidate reactant graphs (shown as candidate reactant graphs

$$“ {\{ {𝒢}_{r , i} \}}_{i = i}^{N} ”$$

108).

In the example shown in FIG. 1, the reactant generation module 102 includes a reaction center identification module 110 and a synthon completion module 112. Table 1 shows an example algorithm implementation for the reactant generation module 102.

TABLE 1

```
Algorithm A1 G²Retro
$Require : {M}_{p} = ( {𝒢}_{p} , {𝒢}_{p}^{B} ) , K , N , max Steps$ [symbol]
    predict top-K synthons
${\{ {s}_{k} , {𝒢}_{s , k} \}}_{k = 1}^{K} = {G}^{2} Retro - RCI ( {𝒢}_{p} , {𝒢}_{p}^{B} , K )$ [symbol]
    predict top-N reactants
${\{ {𝒢}_{r , i} \}}_{i = 1}^{N} = {G}^{2} Retro - SC ( {𝒢}_{p} , {\{ {s}_{k} , {𝒢}_{s , k} \}}_{k = 1}^{K} , N , max Steps )$
$return {\{ {𝒢}_{r , i} \}}_{i = 1}^{N}$
```

As shown in the example, the reactant generation module 102 is configured to invoke two main algorithm function calls (“G²Retro-RCI” (e.g., 110) and “G²Retro-SC” (e.g., 112)) that operate on molecular graphs (e.g., product graph “ [symbol] ₚ” 106a and BRICS graph

$$“ {𝒢}_{p}^{B} ”$$

106b, and/or other graphs described herein). The reaction center identification module 110 (e.g., per algorithm function call “G²Retro-RCI”) is configured to generate a set of candidate synthon graphs along with a corresponding set of log-likelihood scores sₖ for them (log-likelihood function refers to a maximum likelihood estimator of a parameter). The reaction center identification module 110 (e.g., per algorithm function call “G²Retro-RCI”) then selects the top-K predicted synthons 114 (shown as synthon graphs

$$“ {\{ {𝒢}_{s , k} \}}_{k = 1}^{K} ” in {\{ {s}_{k} , {𝒢}_{s , k} \}}_{k = 1}^{K}$$

114) based on the log-likelihood scores sₖ 115, where

$${\{ {𝒢}_{s , k} \}}_{k = 1}^{K}$$

refers to a set of K number of top synthon graphs, indexed by the value k). The K parameter 116 defines the size of the top predictions, i.e., top-K, to be generated.

The synthon completion module 112 (e.g., per function “G²Retro-SC”) is configured to generate a set of top-N reactants

$$( e . g . , “ {\{ {𝒢}_{r , i} \}}_{i = 1}^{N} ”$$

108, shown as 108′) using the generated top-K predicted synthons

$$( e . g . , “ {\{ {𝒢}_{s , k} \}}_{k = 1}^{K} ”$$

shown in 114), e.g., generated from the RCI module 110 where

$${\{ {𝒢}_{r , i} \}}_{i = 1}^{N}$$

refers to a set of N number of top reactant graphs, indexed by the value i). The N parameter 118 defines the size of the top predictions, i.e., top-N, to be generated, the maxsteps parameter 120 refers to the number of iterative steps that the synthon completion module 112 can perform to generate the product reactants, and [symbol] ₚ 106a′ refers to the product graph (e.g., 106a).

### Example Reaction Center Identification (RCI) Operation and Module

The reaction center identification module 110 (e.g., per algorithm function call “G²Retro-RCI”) is configured to identify the reaction centers to generate a set (shown as

$${\{ {s}_{k} , {𝒢}_{s , k} \}}_{k = 1}^{K} ” )$$

of candidate synthon graphs along with a corresponding set of log-likelihood scores (e.g., sₖ 115) for them and then selects the top-K predicted synthons

$$( e . g . , {\{ {𝒢}_{s , k} \}}_{k = 1}^{K}$$

shown in 114).

The reaction center identification module 110 may employ graph-based neural networks that can learn from one or more molecular graphs of a given target product. Each of the molecular graphs can encode the molecule structures in different representation (e.g., product graph “ [symbol] ₚ” 106a and BRICS graph

$$“ {𝒢}_{p}^{B} ”$$

106b, and/or other graphs described herein) to facilitate a multi-path operation for the identification of the reaction center types. The different molecular graphs, when applicable, can be employed to capture the molecular structures from different representations, which can assist in the determination of the candidate synthesis reactants that optimize their synthesis feasibility. For example, in some embodiments, the molecular graphs include a molecular graph representation (e.g., product graph “ [symbol] ₚ” 106a). In other embodiments, the molecular graphs include a molecular representation based on the BRICS model (e.g., BRICS graph

$$“ {𝒢}_{p}^{B} ”$$

106b) that can represent a molecule in terms of fragments. A “fragment” refers to a minimum standard group of atoms that can substitute for an atom or bond in a given molecule. Fragments do not appear in nature and are a lexicon construction to classify such minimum standard groups of atoms. In contrast, a “synthon” refers to a hypothetical unit within a target molecule that represents a potential starting reagent in the retroactive synthesis of that target molecule.

In the example shown in FIG. 1, the retrosynthesis prediction system 100 includes a graph representation learning module 122 (shown “Graph Representation Learning, G²RetroEncoder” module 122) that employs deep neural networks configured to predict for each type of the reaction centers with high accuracies (later discussed herein).

The reaction center identification module (e.g., 110) is configured to (i) use or invoke learning for molecule representation, (ii) select the top-K reaction centers for the set of assessed reaction centers (e.g., bond formation center (“BF-center”), bond type change center (“BC-center”), atom reaction center “A-center,” or a combination there, e.g., multiple new bond formation centers and/or centers having multiple bond type changes, etc.), (iii) select the top-K reaction centers among the set of evaluated assessed reaction centers, and (iv) generating the top-K predicted synthons (e.g.,

$${\{ {𝒢}_{s , k} \}}_{k = 1}^{K}$$

shown in 114) by converting the intermediate products from the assessment to synthons.

The bond formation center (BF-center) refers to a new bond bᵢⱼ that can be formed across the reactants during the reaction but does not exist in the reactants. The bond type change center (BC-center) refers to an existing bond bᵢⱼ in a reactant whose type can change during the reaction due to the gain or loss of hydrogens, while no other changes (e.g., new bond formation) happen. The atom reaction center (A-center) refers to an atom in a reactant from which a fragment is removed during the reaction without new bond formation or bond type changes. From a conducted study, based on a given set of training data sets derived from a database, it was observed in the study that these three types of reaction centers cover 97.7% of the training set, while the remaining 2.3% of the reactions in the training data involve multiple new bond formations or bond type changes.

Nomenclatures. A synthetic reaction can involve a set of reactants {Mᵣ} and a product molecule Mₚ (interchangeably referred to as a target molecule) that is synthesized from the reactants or catalysts [2]. Each reactant Mᵣ has a corresponding synthon MS representing the substructures of Mᵣ that appear in product molecule MP. The product molecule Mₚ can be represented using a product-molecule molecular graph

$${𝒢}_{P}^{M} ,$$

denoted as

$${𝒢}_{P}^{M} = ( 𝒜 , B ) ,$$

where atom set [symbol] is the set of atoms {aᵢ} in the product molecule Mₚ, and bonds B is the set of corresponding bonds {bᵢⱼ}, where bᵢⱼ connects atoms aᵢ and aⱼ. The set of the reactants {Mᵣ} or the set of synthons {Mₛ} of the product molecule Mₚ can be represented using only one molecular graph, the reactant molecular graph

$${𝒢}_{r}^{M}$$

or synthon molecular graph

$${𝒢}_{r}^{M}$$

respectively. The reactant molecular graph

$${𝒢}_{s}^{M} ,$$

and the synthon molecular graph

$${𝒢}_{s}^{M}$$

could be disconnected, with each connected component representing one reactant or one synthon. The superscript M is not shown when there is no ambiguity as it indicates molecules.

Table 2 shows a summary of a set of notations.

TABLE 2

| Notation | Meaning |
|---|---|
| Mᵣ/Mₛ/Mₚ | reactant/synthon/product molecule |
| [symbol] = ( [symbol] , [symbol] ) | molecular graph with atoms [symbol] and bonds [symbol] |
| α | an atom in [symbol] |
| bᵢⱼ | a bond in [symbol] connecting αᵢ and αⱼ |
| [symbol] ^{B} = ( [symbol] ,ε) | BRICS graph with BRICS fragments [symbol] and edges ε |
| n | a BRICS fragment in [symbol] ^{B} |
| eᵤᵥ | an edge connecting two BRICS fragment |
| x | a feature vector for an atom or a bond |
| C_{BF} | a set of bonds neighboring the bond formation center |
| C_{A} | a set of atoms within the reaction center |
| z | a substructure used to complete synthons into reactants |
| [symbol] | a vocabulary with all the substructures in the dataset |

For synthon completion, a substructure z as a bond (i.e., z=bᵢⱼ) or a ring structure (i.e., z={bᵢⱼ|aᵢ, aⱼ∈a single or polycyclic ring}) can be defined as is used to complete synthons into reactants. A substructure vocabulary [symbol] ={z}can be constructed by comparing reactant graphs [symbol] ᵣ's and their corresponding synthon graphs [symbol] ₛ's in a training data set, all the possible substructures can be extracted from their differences.

The Breaking Retrosynthetically Interesting Chemical Substructures (BRICS) fragmentation model [38] can break synthetically accessible bonds in a product molecule Mₚ, following a set of fragmentation rules. Product molecule Mₚ can be represented as a BRICS graph

$${𝒢}_{p}^{B} = ( 𝒱 , ℰ ) ,$$

where each node nᵤ∈ [symbol] represents a BRICS fragment with all the atoms and bonds belonging to it, ε includes synthetically accessible bonds (ε tend to be the reaction centers), and each edge eᵤᵥ∈ε corresponds to a bond bᵢⱼ that connects two BRICS fragments nᵤ and nᵥ (i.e., aᵢ∈nᵤ and aⱼ∈nᵥ). In the dataset, two BRICS fragments may be connected through only one bond. Compared to product-molecule molecular graph

$${𝒢}_{p}^{M} ,$$

BRICS graph

$${𝒢}_{p}^{B}$$

incorporates fragment-level structures of the product molecule Mₚ. For simplicity, when no ambiguity arises, we omit the super/sub-scripts and use molecular graph [symbol] or BRICS graph

$${𝒢}_{p}^{B}$$

to represent [symbol] ^{M} or

$${𝒢}_{p}^{B} ,$$

respectively.

TABLE 3
Algorithm A2 G²Retro-RCI

Require: [symbol] ₚ, [symbol] ^{B}, K

| | [symbol] learn molecule representations |
|---|---|
| 1: | (aᵢ), (bᵢⱼ), hₚ = G²etro-encoder( [symbol] ₚ, [symbol] ^{B}) |
| | [symbol] select top-K BF-centers (Equation 8) |
| 2: | ${\{ {s}^{b} ( {b}_{ij} ) \}}^{K} = top ( K , findCenter ( BF - center , \{ {b}_{ij} \} , {h}_{p} ) )$ |
| | [symbol] select top-K BC-centers (Equation 9) |
| 3: | ${\{ {s}_{k}^{a} ( {b}_{ij} ) \}}^{K} = top ( K , findCenter ( BC - center , \{ {b}_{ij} \} , {h}_{p} ) )$ |
| | [symbol] select top-K A-centers (Equation 10) |
| 4: | ${\{ {s}^{a} ( {a}_{i} ) \}}^{K} = top ( K , findCenter ( A - center , \{ {a}_{i} \} , {h}_{p} ) )$ |
| | [symbol] select top-K centers {Cₖ} and calculate their log-likelihoods |
| | {sₖ} |
| 5: | ${\{ {s}_{k} , {C}_{k} \}}_{k = 1}^{K} = top ( K , {\{ {s}^{b} ( {b}_{ij} ) \}}^{K} , {\{ {s}_{k}^{c} ( {b}_{ij} ) \}}^{K} , {\{ {s}^{a} ( {a}_{i} ) \}}^{K} )$ |
| | [symbol] convert a product into K sets of synthons and update their log- |
| | likelihoods |
| 6: | ${\{ {s}_{k} , {𝒢}_{s , k} \}}_{k = 1}^{K} = {G}^{2} Retro - p 2 s - T ( {𝒢}_{p} , {\{ {s}_{k} , {C}_{k} \}}_{k = 1}^{K} , {h}_{p} )$ |
| 7: | $return {\{ {s}_{k} , {𝒢}_{s , k} \}}_{k = 1}^{K}$ |

In the example, the reaction center identification module 110 is configured to invoke six algorithm function calls: (i) the “G2Retro-encoder” algorithm (Table 3, line 1) to perform learning for molecule representation (e.g., on product graph [symbol] ₚ 106a and BRICS graph

$$“ {𝒢}_{p}^{B} ”$$

106b), (ii) three algorithm calls (Table 3, lines 2, 3, 4) to select the top-K reaction centers for the set of assessed reaction centers (e.g., “BF-center,” “BC-center,” and “A-center”), (iii) an algorithm call (Table 3, line 5) to select the top-K reaction centers among the set of evaluated assessed reaction centers, and (iv) the “G²Retro-p2s-T” algorithm call (Table 3, line 6) to generate the top-K predicted synthons (e. g.,

$${\{ {𝒢}_{s , k} \}}_{k = 1}^{K}$$

shown in 114) by converting the intermediate products from the assessment to synthons.

### A. Molecule Representation Learning

The graph representation learning module 122 may employ deep neural networks to establish learning to predict the types of the reaction centers for a given product molecule. The graph representation learning module 122 may be connected to a data store 123, e.g., housing a set of molecule databases such as the USPTO-50K database described in Yan et al. [12] or other databases described herein.

The learning per module 122 using the data from data store 123 can be subsequently used in the reaction center identification 110 to predict and break a molecular product at the reaction centers. In the example shown in FIG. 1, for a given product molecule Mₚ, the graph representation learning module 122 is configured to learn atom representations over the molecular graph [symbol] using message-passing networks (MPN) 124 (shown as “G²Retro (GMPN)” 124a). For a given product molecule Mₚ, the graph representation learning module (e.g., 122) may also learn the representation of the BRICS fragments over the BRICS graph [symbol] ^{B} (for BRICS fragments) using message passing networks 124 (shown as “G²Retro-B (FMPN)” 124b).

Table 4 shows an example of the G²RetroEncoder graph representation learning module (e.g., 122).

TABLE 4

```
Algorithm A3 G²Retro-encoder
Require: [symbol]   , [symbol]   ^{B} [symbol]
      calculate atom embeddings
1: {aᵢ} = GMPN( [symbol]   ) [symbol]
      calculate the graph embedding (Equation 3)
2: h = Σₐ_{ᵢ}_{∈} [symbol]  ^{a}^{ᵢ},
2. if use BRICS then
        {nᵤ} = BMPN( [symbol]   ^{B}, {aᵢ}) [symbol]
          update the embedding of each atom with its BRICS fragment embedding (Equation 6)
        {aᵢ} = {V (aᵢ ⊕ nᵤ)}
    end if [symbol]
      calculate bond embeddings (Equation 7)
7: {bᵢⱼ} = bondEmb({aᵢ})
8: return {aᵢ}, {bᵢⱼ}, h
```

In the example, the graph representation learning module 122 is configured to (i) calculate atom embeddings (Table 4, line 1), (ii) calculate the BRICS graph fragment embeddings (Table 4, lines 2-6), and calculate the bond embeddings (Table 4, line 7) to generate a set of atom embeddings {aᵢ} in the product molecule Mₚ, and a set of corresponding bond embeddings {bᵢⱼ}, where bᵢⱼ connects atoms aᵢ and aⱼ. The parameter h is the sum of all the atom embeddings.

Another graph representation, junction tree model graphs, may be used in message-passing networks (TMPN); in an alternative to BRICS fragments. Further description of TMPN is provided in Chen et al. [36]., which is incorporated by reference herein in its entirety.

Atom Embedding over Molecular Graphs (GMPN): To calculate the atom embeddings, e.g., per Table 4, line 1, The “G²Retro (GMPN)” graph representation learning module 122 may be configured to learn atom embeddings to capture the atom types and their local neighborhood structures using message passing networks (MPN) by passing messages along with bonds in a given molecular graph (e.g., product molecule graph [symbol] ₚ and synthon graph [symbol] ₛ). In MPN, each bond bᵢⱼ may be associated with two message vectors mᵢⱼ and mⱼᵢ. The message

$${m}_{ij}^{( t )}$$

at t-th iteration may encode the messages passing from aᵢ to aⱼ, per Equation 1:

$${m}_{ij}^{( t )} = {W}_{1}^{a} ReLU ( {W}_{2}^{a} {x}_{i} + {W}_{3}^{a} {x}_{ij} + {W}_{4}^{a} {\sum }_{{a}_{k} ∈ 𝒩 ( {a}_{i} ) \backslash \{ {a}_{j} \}} {m}_{ki}^{( t - 1 )} ) \qquad ( Eq . 1 )$$

where xᵢ is the atom feature vector, including the atom type, valence, charge, the number of hydrogens, whether the atom is included in a ring, and whether the ring is aromatic; xᵢⱼ is the bond feature vector, including the bond type, whether the bond is conjugated or aromatic, and whether the bond is in a ring;

$${W}_{i}^{a ,} s ( i = 1 , 2 , 3 , 4 )$$

are the learnable parameter matrices. The initial message

$${m}_{ij}^{( 0 )}$$

may be initialized with the zero vector; [symbol] (aᵢ) is the set with all the neighbors of aᵢ (i.e., atoms connected with aᵢ); and ReLU is the activation function. The message

$${m}_{ij}^{( t )}$$

can capture or represent some aspects the structure of t-hop neighbors passing through the bond bᵢⱼ to aᵢ, by iteratively aggregating the neighborhood messages

$${m}_{ki}^{( t - 1 )} .$$

With the maximum tₐ iterations, the atom embedding aᵢ may be derived per Equation 2:

$${a}_{i} = {U}_{1}^{a} ReLU ( {U}_{2}^{a} {x}_{i} + {U}_{3}^{a} {\sum }_{{a}_{k} ∈ 𝒩 ( {a}_{i} )} {m}_{k i}^{( 1 … {t}_{a} )} ) \qquad ( Eq . 2 )$$

where

$${m}_{ki}^{( 1 … {t}_{a} )}$$

denotes the concatenation of

$${\{ {m}_{k i}^{( t )} \vert t ϵ [ 1 : {t}_{a} ] \}}_{2} ; {U}_{i}^{a ′} s ( i = 1 , 2 , 3 )$$

are the learnable parameter matrices. To calculate the graph embeddings (Table 4, line 2), the embedding h of the molecular graph [symbol] may be calculated by summing over all the atom embeddings per Equation 3:

$$h = {\sum }_{{a}_{i} ∈ 𝒢} {a}_{i} \qquad ( Eq . 3 )$$

The product molecule embedding hₚ is generated from the product molecular graph [symbol] ₚ, and the synthon embedding hₛ is generated from the synthon graph [symbol] ₛ.

BRICS Fragment Embedding over BRICS Graphs (FMPN): To calculate the graph embeddings (Table 4, lines 3-6), G²Retro-B graph representation learning module 122 may be configured to generate BRICS fragment embeddings by passing the messages along with the connections over BRICS fragments in the BRICS graphs [symbol] ^{B}, e.g., in a similar way as for atom embeddings over molecular graphs [symbol] ₚ. Specifically, each edge eᵤᵥ in the BRICS graphs [symbol] ^{B} is associated with two message vectors eᵤᵥ and eᵥᵤ. The message

$${e}_{u v}^{( t )}$$

at t-th iteration may be updated per Equation 4:

$${e}_{u v}^{( t )} = {W}_{1}^{e} ReLU ( {W}_{2}^{e} {s}_{u} + {W}_{3}^{e} {s}_{uv} + {W}_{4}^{e} {\sum }_{{n}_{w} ∈ 𝒩 ( {n}_{u} ) ∖ \{ {n}_{v} \}} {e}_{w u}^{( t - 1 )} ) \qquad ( Eq . 4 )$$

where sᵤ=Σₐ_{ᵢ}_{∈n}_{ᵤ} aᵢ is an aggregation of the embeddings of all the atoms within the fragment nᵤ; sᵤᵥ=aᵢ is the embedding of atom aᵢ in fragment nᵤ that is included in the edge eᵤᵥ;

$${W}_{i}^{e ′} s ( i = 1 , 2 , 3 , 4 )$$

are the learnable parameter matrices; and the initial message

$${e}_{u v}^{( 0 )}$$

is initialized with a zero vector. The message

$${e}_{u v}^{( t )}$$

may encode the information passing through the edge eᵤᵥ to nᵥ, and thus is used to further derive the embedding of fragment nᵥ per Equation 5:

$${n}_{v} = {U}_{1}^{e} ReLU ( {U}_{2}^{e} {s}_{v} + {U}_{3}^{e} {\sum }_{{n}_{w} ∈ 𝒩 ( {n}_{u} )} {e}_{wv}^{( 1 … {t}_{e} )} ) \qquad ( Eq . 5 )$$

where

$${e}_{wv}^{( 1 … {t}_{e} )}$$

denotes the concatenation of

$$\{ {e}_{w v}^{( t )} \vert t ϵ [ 1 : {t}_{e} ] \} ; {U}_{i}^{e ′} s ( i = 1 , 2 , 3 )$$

are the learnable parameter matrices. With [symbol] ^{B}, G²Retro-B graph representation learning module 122 can enrich the representation of atom aᵢ with the embedding nᵥ of the fragment that aᵢ belongs to.

The enriched atom representation may be denoted as Equation 6:

$${a}_{i}^{′} = V ( {a}_{i} ⊕ {n}_{v} ) \qquad ( Eq . 6 )$$

where V is a learnable hyperparameter matrix; ⊕ represents the concatenation operation.

### Example Reaction Center Prediction Operation and Module

Referring back to the example algorithm for the reaction center identification module (e.g., 110), following the learning for molecule representation, the algorithm function calls: (i) the three algorithm calls (Table 3, lines 2, 3, 4) (e.g., per Module 125) to select the top-K reaction centers for the set of assessed reaction centers (e.g., “BF-center,” “BC-center,” and “A-center”), then (ii) an algorithm call (Table 3, line 5) to select the top-K reaction centers among the set of evaluated assessed reaction centers, and then (iii) the “G²Retro-p2s-T” algorithm call (Table 3, line 6) to generate the top-K predicted synthons (e.g., shown in 114) by converting the intermediate products from the assessment to synthons. The various reaction center modules 126, 128, 130 are trained via a neural network 131 (shown as “RC Score Training” 131).

The RC Score Training module 131 may be connected to a data store (shown as 135), e.g., housing a set of molecule databases such as the USPTO-50K database described in Yan et al. [12] or other databases described herein. The data stores 135 and 123 may be the same.

Reaction Centers with New Bond Formation (BF-center): To calculate the bond embeddings, e.g., per Table 4, line 7, to generate a set of atoms {aᵢ} in the product molecule Mₚ, and a set of corresponding bonds {bᵢⱼ}, where bᵢⱼ connects atoms aᵢ and aⱼ, following Somnath et al. [7], the reaction center score training module 131 may derive the bond representations per Equation 7:

$${b}_{ij} = {U}_{1}^{b} ReLU ( {U}_{2}^{b} {x}_{i j} + {U}_{3}^{b} ( {a}_{i} + {a}_{j} ) + {U}_{4}^{b} Abs ( {a}_{i} - {a}_{j} ) ) \qquad ( Eq . 7 )$$

where Abs(⋅) represents the absolute difference;

$${U}_{i}^{b ′} s ( i = 1 , 2 , 3 , 4 )$$

are the learnable parameter matrices. The graph representation learning module 122 may use the sum and the absolute difference of embeddings of the connected atoms to capture the local neighborhood structure of bond bᵢⱼ. Meanwhile, the two terms are both permutation-invariant to the order of aᵢ and aⱼ, and, together, can differentiate the information in aᵢ and aⱼ. In the case of G²Retro-B, the bond representations may be derived by replacing the atom embeddings aᵢ and aⱼ with the enriched atom representations

$${a}_{i}^{′} and {a}_{\dot{i}}^{′}$$

calculated as in Equation 6.

With the determined bond representation, the reaction center identification module 110 (per Module 126) may calculate a score (to select the top-K BK-centers per Table 3, line 2) for each bond bᵢⱼ using Equation 8:

$${s}^{b} ( {b}_{i j} ) = {q}^{b} ReLU ( {Q}_{1}^{b} {b}_{i j} + {Q}_{2}^{b} {h}_{p} ) \qquad ( Eq . 8 )$$

where hₚ is the representation of the product graph [symbol] ₚ calculated as in Equation 3; q^{b} is a learnable parameter vector and

$${Q}_{1}^{b} and {Q}_{2}^{b}$$

are the learnable parameter matrices. The reaction center identification module 110 can measure the likelihood the bond bᵢⱼ is a BF-center by evaluating the bond itself (i.e., bᵢⱼ) and the structure of the entire product graph (i.e., hₚ). The reaction center identification module 110 may score each bond in Mₚ and select the most possible BF-center candidates {bᵢⱼ} with the highest scores. The reaction center identification module 110 may break each product at each possible bond reaction center into synthons, and thus can generate multiple possible reactions.

To learn the learnable parameter vectors and matrices,

$${Q}_{1}^{b} , {Q}_{2}^{b} ,$$

and q^{b}, the reaction center identification module 110 employs the neural network training module 131.

The function “findCenter” for BF-centers (Table 3, line 2) may be defined per Equation 8 using the set of the most possible BF-center candidates {bᵢⱼ} and the product molecule embedding hₚ. The function “top” can rank and select the top number of K results from the scored output of the findCenter function to provide the top-K selected set of scores {s^{b}(bᵢⱼ)}^{K} (shown as 126′) and the corresponding bond bᵢⱼ.

Reaction Centers with Bond Type Change (BC-center): For reaction center associated with bond type change without new bond formations, the reaction center identification module 110 (per Module 128) may calculate (to select the top-K BC centers per Table 3, line 3) a score vector s^{c}∈ [symbol] ^{1×3} for each bond bi; in Mₚ per Equation 9:

$${s}^{c} ( {b}_{i j} ) = {Q}_{1}^{c} ReLU ( {Q}_{2}^{c} {b}_{i j} + {Q}_{3}^{c} {h}_{p} ) \qquad ( Eq . 9 )$$

where

$${Q}_{i}^{c ,} s ( i = 1 , 2 , 3 )$$

are the learnable parameter matrices. Each element in the score vector s^{c} may represent if bᵢⱼ is the BC-center, the score of bᵢⱼ's original type in [symbol] ᵣ being single, double, and triple bond, respectively. The element in the score vector s^{c} corresponding to bᵢⱼ's types in the product molecule graph [symbol] ₚ may be reset to “0” (i.e., the bᵢⱼ's type has to be different in the reactant graph [symbol] ᵣ compared to that in product molecule graph [symbol] ₚ). Subsequently, the reaction center identification module 110 may select the most possible BC-center candidates {bᵢⱼ} and their possible original bond types scored by the s^{c}(⋅). The reaction center identification module 110 may then change the corresponding bond type to construct the appropriate synthons.

To learn the learnable parameter matrices,

$${Q}_{i}^{c} ,$$

the reaction center identification module 110 employs the neural network training module 131.

The function “findCenter” for BC-centers (Table 3, line 3) may be defined per Equation 9 using the set of the most possible BC-center candidates {bᵢⱼ} and the product molecule embedding hₚ. The function “top” can rank and select the top number of K results from the scored output of the findCenter function to provide the top-K selected set of BC-center score

$${\{ {s}_{k}^{c} ( {b}_{i j} ) \}}^{K}$$

(shown as 128′) and the corresponding bond bᵢⱼ.

Reaction Centers with Single Atoms (A-center): As part of the analysis to select the top-K reaction centers for the A-center reaction center, the reaction center identification module 110 may determine a center score for each atom aᵢ in a product molecule Mₚ per Equation 10:

$${s}^{a} ( {a}_{i} ) = {q}^{a} ReLU ( {Q}_{1}^{a} {a}_{i} + {Q}_{2}^{a} {h}_{p} ) \qquad ( Eq . 10 )$$

where q^{a} is a learnable parameter vector, and

$${Q}_{1}^{a} and {Q}_{2}^{a}$$

the learnable parameter matrices. The reaction center identification module 110 may select the set of atoms {aᵢ} in the product molecule Mₚ with the highest scores as potential A-center's. In the synthon completion operation, the new fragments may be attached to the atom reaction centers.

To learn the learnable parameter vectors and matrices

$${Q}_{1}^{a} , {Q}_{2}^{a} ,$$

and q^{a}, the reaction center identification module 110 per the neural network training module 131 can per a minimization operation of a cross-entropy loss per Equation 11, e.g., in combination with the BF-center, A-center, and BC-center score training.

With the scores of the three types of reaction centers, the training module 131 can minimize the cross entropy loss of Equation 11 to learn the scoring functions, e.g., for Equations 8, 9, and 10:

$${ℒ}^{s} = - {\sum }_{{b}_{i j} ∈ ℬ} ( {y}_{i j}^{b} {l}^{b} ( {b}_{i j} ) + {\sum }_{k = 1}^{3} {\text{?}}_{k} ( {y}_{i j}^{c} ) {l}_{k}^{c} ( {b}_{i j} ) ) - {\sum }_{{a}_{i j} ∈ 𝒜} {y}_{i}^{a} {l}^{a} ( {a}_{i} ) \qquad ( Eq . 11 )$$

where y*(x=a, b, c) is the label indicating whether the corresponding candidate is the ground-truth reaction center of type * (y*=1) or not (y*=0); [symbol] ₖ(x) is an indicator function ( [symbol] ₖ(x)=1 if x=k, 0 otherwise), and thus [symbol] ₖ(yᵤ_{ⱼ}^{c}) indicates whether the ground-truth bond type of bᵢⱼ is k or not (k=1, 2, 3 indicating single, double or triple bond); and l*(⋅) (*=a, b, c) is the probability calculated by normalizing the

$${l}^{*} ( \cdot ) = exp ( {s}^{*} ( \cdot ) ) / Δ , where Δ = {\sum }_{{b}_{i j} ∈ ℬ} ( exp ( {s}^{b} ( {b}_{i j} ) ) + {\sum }_{k = 1}^{3} exp ( {s}_{k}^{c} ( {b}_{i j} ) ) ) + {\sum }_{{a}_{i} ∈ 𝒜} exp ( {s}^{a} ( {a}_{i} ) ) ( {l}_{k}^{c} ( x ) = exp ( {s}_{k}^{c} ( x ) ) / Δ ) .$$

The function “findCenter” for A-centers (Table 3, line 4) may be defined per Equation 10 using the set of the most possible A-center candidates {aᵢ} and the product molecule embedding hₚ. The function “top” can rank and select the top number of K results from the scored output of the findCenter function to provide the top-K selected set of A-center scores {s^{a}(aᵢ)}^{K} (shown as 130′) and the corresponding atom aᵢ.

Select Reaction Center Top-K: Following the calculation of the top-K reaction centers for each reaction center type, the reaction center identification module 110 is configured, e.g., per Module 132, to then select the top-K reaction centers. In Table 3, line 5, the function “top” can rank and select the top number of K results

$$( {\{ {s}_{k} , {C}_{k} \}}_{k = 1}^{K} )$$

from the previously calculated top-Ks, e.g., top-K selected set of scores {s^{b}(bᵢⱼ)}^{K}(126′), the top-K selected set of BC-center scores

$${\{ {s}_{k}^{c} ( {b}_{i j} ) \}}^{K}$$

(128′), and top-K selected set of A-center scores {s^{a}(aᵢ)}^{K} (130′) and associated intermediate results.

Product-Synthon Transformations (P2S-T): Following the selection of the top-K reaction centers and associated intermediate results

$$( e . g . , {\{ {C}_{k} \}}_{k = 1}^{K}$$

shown in 133), the reaction center identification module 110 per the product-synthon transformation module 134 is configured to convert if needed, the top-K intermediate products to a set of synthons

$$( e . g . , {\{ {𝒢}_{s , k} \}}_{k = 1}^{K}$$

shown in 114), e.g., to be used by the synthon competition module 112.

Table 5 shows an example implementation of the product-synthon transformation module (e.g., 134). The product-synthon transformation module (e.g., 134) is configured to transform the format of the top-K intermediate products generated in the prior analysis to synthon graphs. The product-synthon transformation module (e.g., 134) can update the scores and/or selected top-K intermediate products by accounting for bond type change when evaluating reaction centers with new bond formations (BF-centers) and/or account for atom charge change when evaluating reaction centers with fragments removed without new bond formation or bond type changes (A-centers).

TABLE 5
Algorithm A4 G²Retro-p2s-T

$Require : {𝒢}_{p} , {\{ {s}_{k} , {C}_{k} \}}_{k = 1}^{K} , {h}_{p}$

| | |
|---|---|
| 1: | for each sₖ, [symbol] ₖ do |
| 2: | if [symbol] ₖ is BF-center then |
| | [symbol] predict bonds with induced type changes and calculate the log-likelihood s_{BF} for the predictions of C_{BF}(Cₖ) |
| 3: | [symbol] _{BF}′ ( [symbol] ₖ), s_{BF} = BTCP( [symbol] ₖ, [symbol] _{BF}( [symbol] ₖ), hₚ) |
| | [symbol] add the predicted bonds with type changes into the center |
| | [symbol] ₖ = [symbol] ₖ ∪ [symbol] _{BF}′( [symbol] ₖ) |
| | [symbol] update the log-likelihood score |
| | sₖ = sₖ + s_{BF} |
| | end if |
| | [symbol] predict atoms with charge changes for all the atoms within the center [symbol] _{A} ( [symbol] ₖ), and calculate the log likelihood |
| | score s_{A} for the predictions of [symbol] _{A} ( [symbol] ₖ) |
| | [symbol] _{A}′ ( [symbol] ₖ), s_{A} = ACP( [symbol] ₖ, [symbol] _{A}( [symbol] ₖ), hₚ) |
| | [symbol] update the log-likelihood score |
| 8: | sₖ = sₖ + s_{A} |
| | [symbol] transform the product graph into the synthon graph |
| 9: | [symbol] _{s,k} = transform( [symbol] ₚ, [symbol] ₖ, [symbol] _{A}′ ( [symbol] ₖ)) |
| 10: | end for |
| 11: | $return {\{ {s}_{k} , {𝒢}_{s , k} \}}_{k = 1}^{K}$ |

In the example of FIG. 1 and Table 5, the product-synthon transformation module 134 is configured to (i) update the scores for selected top-K intermediate products involving BF-centers by predicting the bonds for a given induced type change (Table 5, lines 2-6) and (ii) update the scores for selected top-K intermediate products involving BF-centers, BC-centers and A-centers by predicting atoms with charge changes (Table 5, lines 7-9). In one implementation, the product-synthon transformation module 134 (i) invokes a BF-Center Induced Bond Type Change Prediction (BTCP) module 136 to predict the bonds for a given induced type change, and (ii) invokes an atom charge prediction (ACP) module 140 to predict the atoms with charge changes.

BF-center Induced Bond Type Change Prediction (BTCP): To take into account scenario in synthetic reactions in which new bonds could induce the changes of neighbor bonds, the reaction center identification module 110 per the BTCP module 136 (e.g., executed by the function, per Table 5, line 3, to add predicted bonds with type changes into the reaction center) may be employed to determine a likelihood that the types of bonds neighboring the BF-center could change during a given reaction. Given the BF-center bᵢⱼ, the BF-center neighbor bonds [symbol] _{BF} for a set of the bonds neighboring bᵢⱼ can be determined per Equation 12:

$${𝒞}_{B F} = \{ {b}_{i k} \vert {a}_{k} ∈ 𝒩 ( {a}_{i} ) ∖ \{ {a}_{j} \} \} ⋃ \{ {b}_{j k} \vert {a}_{k} ∈ 𝒩 ( {a}_{j} ) ∖ \{ {a}_{i} \} \} \qquad ( Eq . 12 )$$

The BTCP module 136 may determine a probability distribution f^{b}∈ [symbol] ^{1×4} for each neighboring bond in [symbol] _{BF}, denoted as b_{i/jk}∈ [symbol] _{BF}, per Equation 13:

$${f}^{b} ( {b}_{i / jk} ) = softmax ( {V}_{1}^{b} {b}_{i / jk} + {V}_{2}^{b} {b}_{i j} + {V}_{3}^{b} {h}_{p} ) \qquad ( Eq . 13 )$$

where f^{b} is the probability distribution,

$${V}_{i}^{{b}^{′}} s ( i = 1 , 2 , 3 )$$

are the learnable parameter matrices. The first element

$${f}_{1}^{b}$$

in f^{b} represents the likelihood the b_{i/jk} type is changed during the reaction (It is determined as type change if

$${f}_{1}^{b}$$

is not the maximum in f^{b}), and the other three represent the likelihood that the original b_{i/jk} in the reactant is a single, double, or triple bond, respectively (these three elements are reset to 0 if b_{i/jk} type is predicted unchanged). The BTCP module 136 may measure the neighbor bond type change by evaluating the neighbor bond itself (i.e., b_{i/jk}), the BF-center (i.e., bᵢⱼ), and the overall product (i.e., hₚ) (e.g., in the “BTCP” function of Table 5, line 3). The reaction center identification module 110 may employ the BF-center neighbor bonds [symbol] _{BF} determination to update the synthons [symbol] ₛ, e.g., to change the neighboring bonds of the BF-center to their predicted original types during the later phase of the synthon generation.

To train the learnable parameter matrices

$${V}_{i}^{{b}^{′}} s$$

of the BTCP module 136, the product-synthon transformation module 134 may include a neural network training module 141 (shown as “Charge Change Training” module 141).

The BTCP module 136 may join (e.g., per Table 5, line 4) the predicted bonds with the induced bond types to the selected top number of K results Cₖ. The BTCP module 136 may update (e.g., per Table 5, line 5) the log-likelihood score sₖ for the selected top number of K results Cₖ.

To learn the learnable parameter matrices,

$${V}_{i}^{{b}^{′}} s ( i = 1 , 2 , 3 ) ,$$

the BTCP module 136 per the neural network training module 131 can learn the predictor f^{b}(⋅) for neighbor bond changes (Equation 13).

Atom Charge Prediction (ACP): To take into account scenario in synthetic reactions in which an atom charge change occurs when a fragment is removed without new bond formation or bond type changes, the reaction center identification module 110 via the Atom Charge Prediction (ACP) module 140 predicts whether the charge of aᵢ remains unchanged in reactants for all the atoms at involved in the reaction center (BF-center, BC-center or A-center) or BF-center changed neighbor bond

$${𝒞}_{BF}^{′}$$

The ACP module 140 may use an embedding c to represent all the involved bond formations and changes in the product-synthon transformation module 134. If the reaction center is predicted as a BF-center at bᵢⱼ, G²Retro calculates the embedding c per Equation 14:

$$c = \text{?} {W}_{1}^{c} R e L U ( {W}_{2}^{c} {x}_{kl}^{′} + {W}_{3}^{c} {b}_{k l} ) \qquad ( Eq . 14 )$$

where,

$${𝒞}_{BF}^{′}$$

is a subset of [symbol] _{BF} with all the bonds that changed types;

$${x}_{kl}^{′}$$

is a 1×4 one-hot vector, in which

$${x}_{kl}^{′} ( 0 ) = 1$$

if bond bₖₗ is the bond formation center (i.e., bₖₗ=bᵢⱼ), or

$${x}_{kl}^{′} ( i ) = 1 ( i = 1 , 2 , 3 )$$

if bₖₗ type is changed from single, double or triple bond in reactants, respectively, during the reaction (i.e., bₖₗ is in

$${\text{?}}_{BF}^{′} ( {b}_{i j} ) ) ; {W}_{i}^{c} ’ s ( i = 1 ; 2 ; 3 )$$

are the learnable parameter matrices. To train the learnable parameter matrices

$${W}_{i}^{c} ’ s$$

of the BTCP module 136, the product-synthon transformation module 134 may employ the charge change training module (e.g., 141).

If the reaction center is predicted as a BC-center at bᵢⱼ, c is calculated per Equation 15:

$$c = {W}_{1}^{c} ReLU ( {W}_{2}^{c} {x}_{kl}^{′} + {W}_{3}^{c} {b}_{i j} ) \qquad ( Eq . 15 )$$

where

$${x}_{ij}^{′} ( 0 ) = 0 and {x}_{ij}^{′} ( i ) ( i = 1 , 2 , 3 )$$

if bₖₗ type is changed from the single, double, or triple bond in reactants, respectively, during the reaction. If the reaction center is an A-center, the product-synthon transformation (e.g., p2s-T) operation is not performed, and c=0.

Electron Change Prediction: With the embedding c for the product-synthon transformation, the ACP module 140 may calculate the probabilities that a, will have charge changes during the reaction per Equation 16:

$${f}^{c} ( {a}_{i} ) = softmax ( {V}_{1}^{c} {a}_{i} + {V}_{2}^{c} c ) \qquad ( Eq . 16 )$$

where

$${V}_{1}^{c} and {V}_{2}^{c}$$

are the learnable parameter matrices; f_{c}∈ [symbol] ^{1×3} is a vector representing the probabilities of accepting one electron, donating one electron or no electron change during the reaction. The option corresponding to the maximum value in f^{c} is selected and will be applied to update synthon charges accordingly. The ACP module 140 may consider at most one electron change since this is the case for all the reactions in the dataset.

To train the learnable parameter matrices

$${V}_{1}^{c} and {V}_{2}^{c}$$

of the ACP module 140, the learnable parameter matrices

$${V}_{i}^{b} ’ s ( i = 1 , 2 , 3 )$$

of the BTCP 136, the reaction center identification module 110 may employ the neural network training module (e.g., 131, 141). The charge change training module 141 may learn the predictor f^{c}(⋅) for atom charge changes (Equation 16) and f^{b}(b_{i/jk}) for BF-center induced bond type changes (Equation 13) by minimizing their respective cross entropy loss [symbol] ^{c} and [symbol] ^{b}. These two losses may be learned together with the cross entropy loss [symbol] ^{s} (Equation 11) for all the reaction centers. Therefore, the center identification module learns the predictors by solving the optimization problem per Equation 17:

$${min}_{Θ} {ℒ}^{s} + {ℒ}^{b} + {ℒ}^{c} \qquad ( 17 )$$

where Θ is the set of all the parameters in the prediction functions.

### Example Synthon Completion (SC) Operation and Module

Once the reaction centers are identified, and all the product-synthon transformations (e.g., via p2s-T module 134) are conducted to generate synthons from products, the synthon completion module 112 is configured to complete the synthons into the reactants by sequentially attaching molecular substructures (e.g., bonds or strings) to the reaction centers of the top-K identified synthons for the reaction, e.g., in an iterative manner from a set of candidate substructures or bonds. Actions involved in the synthon completion operation (completion process) may be referred to as synthon-reactant transformations (s2r-T).

During the completion process, the intermediate molecules {M*} may be represented as molecular graph { [symbol] *}. At step t, the atom in the intermediate molecular graph [symbol] *^{(t)} ( [symbol] *⁽⁰⁾= [symbol] ₛ) that new substructures will be attached to may be denoted as atom a^{(t)} and the substructure attached to a^{(t)} may be denoted as z^{(t)}, resulting in [symbol] *₍ₜ₊₁₎.

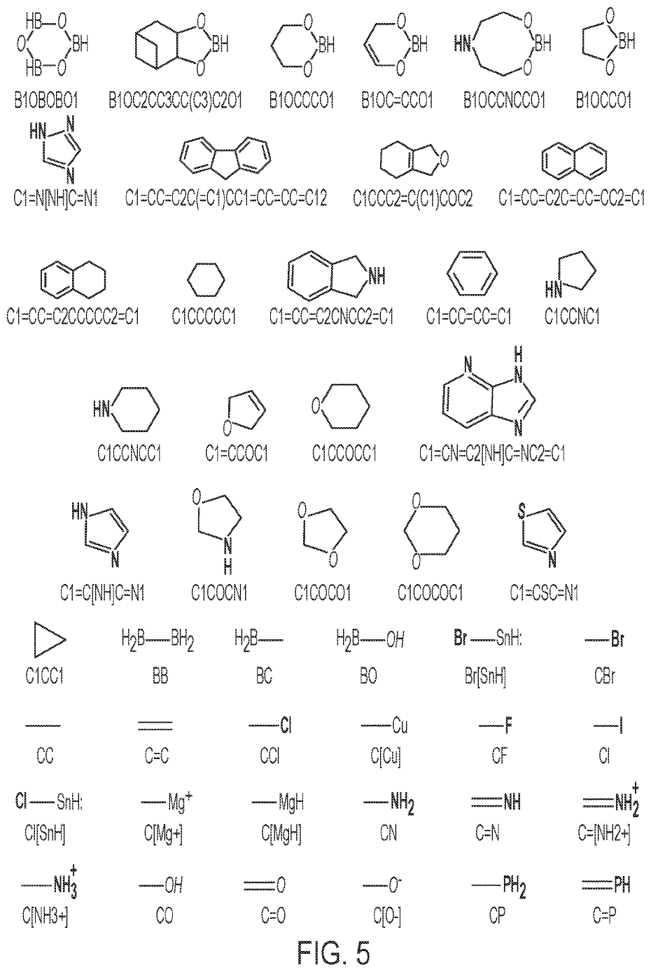

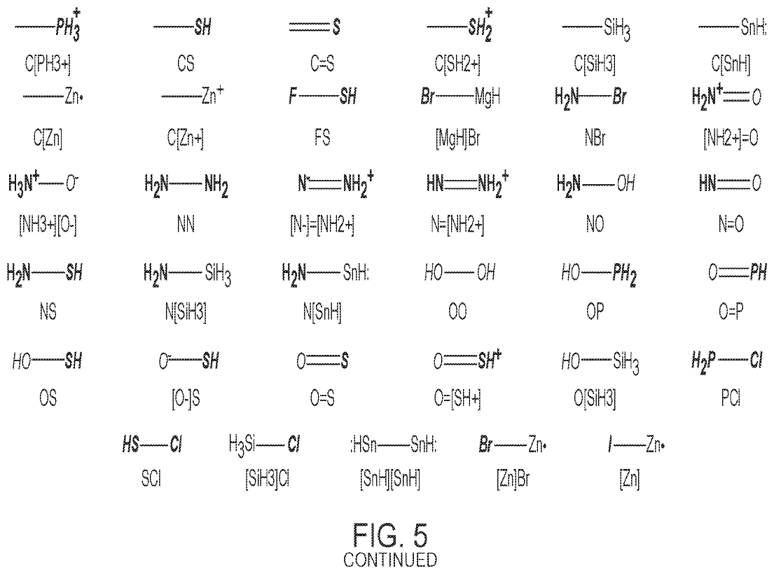

The candidate substructures may be generated and stored using one or more machine learning or artificial intelligence algorithms. In some embodiments, a set of candidate substructures are generated through training using data from a public database of molecules. In an implementation, at least 83 candidate substructures are used that are generated from the USPTO-50K database described in Yan et al. [12] or other databases described herein. FIG. 5 shows an example set of the 83 substructures that may be used by the exemplary completion module to complete synthons.

The synthon completion module 112 may be executed as the second part for a semi-template-based method. The exemplary system and method can complete an updated synthons from the synthons generated by the reaction center identification module 110. In other embodiments, the exemplary system and method can complete the synthons into reactants using synthons and other intermediate or hypothetical molecular units from one or more other databases using the disclosed method.

The synthon completion module 112 is configured to, for each candidate synthon (e.g., 114), iteratively attach substructures to each synthon through a searching algorithm, e.g., beam search, or other greedy algorithms, that can prioritize the most possible reactants along the synthon completion paths.

Table 6 shows an example algorithm implementation for the synthon completion module 112. In the example of FIG. 1 and Table 6, the synthon completion module 112 is configured to receive the top-K predicted synthons and associated scores

$${\{ {s}_{k} , {𝒢}_{s , k} \}}_{k = 1}^{K}$$

(114, 115), the product graph “ [symbol] ₚ” (e.g., 106a, 106b), N parameter 118, and the maxsteps parameter 120. In Table 6, the synthon completion module 112 may execute the G²Retro-Encoder function to learn the molecule representations hₚ and

$${\{ {h}_{s , k} \}}_{k = 1}^{K}$$

from the product molecule graph [symbol] ₚ (e.g., 106′) and the top-K predicted synthons

$${\{ {𝒢}_{s , k} \}}_{k = 1}^{K}$$

(shown in 114).

The synthon completion module 112 then initialize (Table 6, line 4) the priority queue for the top-K predicted synthons

$${\{ {𝒢}_{s , k} \}}_{k = 1}^{K}$$

along with a corresponding priority value

$${\{ {s}_{k} \}}_{k = 1}^{K} .$$

And, while the priority queue is not empty (Table 6, line 6) and the current step t is less than the maxsteps value, then execute the beam search function “G²Retro-beam-search”.

TABLE 6
Algorithm A5 G/Retro-SC

$Require : {𝒢}_{p} , {\{ {s}_{k} , {𝒢}_{s , k} \}}_{k = 1}^{K} , N , max Steps$

| | |
|---|---|
| 1: | t = 0 |
| | [symbol] learn molecule representations |
| 2: | -. - , hₚ = G²Retro-encoder( [symbol] ₚ) |
| 3: | $- , - , {\{ {h}_{s , k} \}}_{k = 1}^{K} = {G}^{2} Retro - encoder ( {\{ {𝒢}_{s , k} \}}_{k = 1}^{K} )$ |
| | $⊳ initialize a priority queue with synthons {\{ {𝒢}_{s , k} \}}_{k = 1}^{K} as elements and {\{ {s}_{k} \}}_{k = 1}^{K} as their priorities$ |
| | ${Q}^{( 0 )} = priorityQueue ( {\{ {s}_{k} , {𝒢}_{s , k} \}}_{k = 1}^{K} )$ |
| | [symbol] initialize an empty priority queue to store complete reactants |
| | R = priority Queue( ) |
| | while IQ^{(t)} .isEmpty( ) and t ≤ maxSteps do |
| | [symbol] stop the completion when it is impossible to get reactants better than the top-N reactants in R |
| 7: | if R.size( ) ≥ N and R.nthLargest Priority(N) ≥ [symbol] ^{(t)} maxPriority( ) then |
| 8: | break |
| 9: | end if |
| | [symbol] complete synthons through boam search |
| 10: | ${Q}^{( t + 1 )} , R = {G}^{2} Retro - beam - search ( {Q}^{( t )} , R , {\{ {h}_{s , k} \}}_{k = 1}^{K} , {h}_{p} , N )$ |
| 11: | t = t + 1 |
| 12: | end while |
| | [symbol] output top-N reactants |
| 13: | ${\{ {𝒢}_{r , i} \}}_{i = 1}^{N} = R \cdot nLargost ( N )$ |
| 14: | $return {\{ {𝒢}_{r , i} \}}_{i = 1}^{N}$ |

### Top-N Reactant Graph Generation—Beam Search

Once the top-K reaction centers for each product are selected, and their synthon graphs are generated, the synthon completion module 112 completes the synthon graphs

$${\{ {𝒢}_{s , k} \}}_{k = 1}^{K}$$

(e.g., 106′) into reactant graphs. During the completion, the synthon completion module 112 scores each possible reactant graph and uses their final scores to select the top-N reactant graphs, and thus top-N most possible synthetic reactions, for each product. Since during synthon completion, the attachment substructure type prediction (Equation 20) gives a distribution of all possible attachment substructures; by using the top possible substructures, each synthon and its intermediate graphs can be extended to multiple different intermediate graphs, leading to exponentially many reactant graphs. The intermediate graphs are denoted as

$${\{ {𝒢}_{i j}^{* ( t )} \}}_{i = 1}^{K} ,$$

$$where {𝒢}_{i j}^{* ( t )}$$

is for the j-th possible intermediate graph of the i-th synthon graph [symbol] _{s,i} at step t. However, to fully generate all the possible completed reactant graphs, excessive computation is demanded. Rather, the synthon completion module 112 applies a greedy beam search algorithm, per module 142, to only explore the most possible top reactant graph completion paths.

Table 7 shows an example algorithm implementation for the synthon beam search module 142.

TABLE 7

| Algorithm AG G²Retro-beam-search | |
|---|---|
| $Require : Q , R , {\{ {h}_{s , k} \}}_{k = 1}^{K} , {h}_{p} , N$ | |
| 1: | I = [symbol] .size( ) |
| 2: | [symbol] ' = priorityQueue( ) |
| 3: | while ! [symbol] .isEmpty( ) do |
| 4: | ${s}_{i} , {𝒢}_{i}^{*} = Q \cdot pop ( )$ |
| | $⊳ get the index of the synthon corresponding to {𝒢}_{i}^{*}$ |
| 5: | $k = {𝒢}_{i}^{*} \cdot getSynthonIdx ( )$ |
| | $⊳ predict the atom attachment for {𝒢}_{i}^{*}$ |
| 6: | ${\{ {s}_{i , j} , {𝒢}_{i , j}^{′} \}}_{j = 1}^{N + 1} = {G}^{2} Retro - AAP ( {𝒢}_{i}^{*} , {s}_{i} , {h}_{s , k} , {h}_{p} , N )$ |
| 7: | end while |
| | [symbol] select top-N intermediate graph candidates |
| 8: | ${\{ {s}_{i} , {𝒢}_{i}^{′} \}}_{i = 1}^{N} = top ( N , {\{ {\{ {s}_{i , j} , {𝒢}_{i , j}^{′} \}}_{j = 1}^{N + 1} \}}_{i = 1}^{I} )$ |
| 9: | $for each {s}_{i} , {𝒢}_{i}^{′} do$ |
| 10: | $if {𝒢}_{i}^{′} \cdot isComplete ( ) then$ |
| 11: | $R \cdot push ( {s}_{i} , {𝒢}_{i}^{′} )$ |
| 12: | else |
| 13: | ${Q}^{′} \cdot push ( {s}_{i} , {𝒢}_{i}^{′} )$ |
| 14: | end if |
| 15: | end for |
| 16: | return [symbol] ′, R |

The beam search module 142 scores each intermediate graph

$${𝒢}_{ij}^{* ( t )}$$

using a score

$${s}_{ij}^{( t )} ,$$

e.g., via the G2Retro-AAP function (Table 7, line 6), which is calculated as the sum over all the log-likelihoods of all the predictions along the completion path from [symbol] ₛ up to

$${𝒢}_{ij}^{* ( t )} ; {s}_{ij}^{( 0 )}$$

is initialized as the sum of the log-likelihoods of all the predictions from [symbol] ₚ to [symbol] ₛ. At each step t (e.g., t≤30), each intermediate graph

$${𝒢}_{ij}^{* ( t )}$$

is extended to at most N+1 intermediate graph candidates. These N+1 candidates include the one that is predicted to stop at the atom that the new substructures could be attached to (i.e., as a^{(t)} in Equation 18 (shown below); this intermediate graph could be further completed at other atoms) in this step, and at most N candidates with the top-N predicted substructures attached (e.g., Equation 20, shown below). Among all the candidates generated from all the intermediate graphs at step t, the top-N-scored ones will be further forwarded into the next completion step 1+1. In case some of the top-N graphs are fully completed, the remaining will go through the next steps. This process will be ended until the number of all the completed reactant graphs at different steps reaches or goes above N. Then, among all the incompleted graphs at the last step, the intermediate graphs with log-likelihood values higher than the N-th largest score in all the completed ones will continue to complete as above. The entire process will end until no more intermediate graphs are qualified to further completion. Among all the completed graphs, the top-N graphs are selected as the generated reactants.

Atom AttachmentPrediction (AAP): Table 8 shows an example algorithm implementation for the atom attachment prediction (AAP) module 144.

TABLE 8
Algorithm A7 G²Retro-AAP

Require: [symbol] *, s, hₛ, hₚ, N

| | [symbol] get the atom that new substructures will be attached to |
|---|---|
| 1: | a = [symbol] * .nextAttachment Point( ) |
| | [symbol] predict whether further attachment should be added to a |
| | (Equation 18) |
| 2: | s^{o}, s^{~o} = AACP(a, hₛ, hₚ) |
| | $⊳ extend {𝒢}^{*} to the candidate {𝒢}_{1}^{′} that is predicted to stop at a$ |
| 3: | ${𝒢}_{1}^{′} = stop ( {𝒢}^{*} , a )$ |
| | $⊳ update the log - likelihood value of {𝒢}_{1}^{′}$ |
| 4: | ${s}_{1}^{′} = s + {s}^{- o}$ |
| | [symbol] predict the top-N new substructure attachments (Equation 21) |
| 5: | ${\{ {z}_{i} , {s}_{i}^{z} \}}_{i = 1}^{N} = top ( N , AATP ( a , {h}_{s} , {h}_{p} ) )$ |
| | $⊳ extend {𝒢}^{*} to the candidates {\{ {𝒢}_{i}^{′} \}}_{i = 2}^{N + 1} with the top - N substructures$ |
| 6: | ${\{ {𝒢}_{i}^{′} \}}_{i = 2}^{N + 1} = attach ( {𝒢}^{*} , {\{ {z}_{i} \}}_{i = 1}^{N} )$ |
| | $⊳ update the log - likelihood values of {\{ {𝒢}_{i}^{′} \}}_{i = 2}^{N + 1}$ |
| 7: | ${{\{ {s}_{i}^{′} \}}_{i = 2}^{N + 1} = ( s + {s}^{o} + {s}_{i}^{z} \}}_{i = 1}^{N}$ |
| 8: | $return {\{ {s}_{i}^{′} , {𝒢}_{i}^{′} \}}_{i = 1}^{N + 1}$ |

In the example, the AAP module 144 is configured to (i) perform an operator 146 (line 1) to get a current atom for a potential attachment, (ii) perform an operator 148 (line 2) to determine a prediction value for an attachment per Equation 18 (shown below) using the AACP module, (iii) perform an operator 150 (line 3) to extend the new substructure [symbol] * to a candidate

$${𝒢}_{1}^{′} ,$$

(iv) perform an operator 152 (line 4) to update the log-likelihood value of the candidate

$${𝒢}_{1}^{′} ,$$

(v) perform an operator 154 (line 5) to determine and select the top-N predictions of the new substructure attachments per Equation 20 (shown below), (vi) perform an operator 156 (line 6) to extend the new substructure [symbol] * to the candidate with the top-N substructures, and (vii) perform an operator 158 (line 7) to update the set of log-likelihood values

$${\{ {s}_{i}^{′} \}}_{i = 2}^{N + 1}$$

for the set of candidate reactants

$${\{ {𝒢}_{i}^{′} \}}_{i = 2}^{N + 1} .$$

### Attachment Continuity Prediction (AACP)

The synthon completion module 112 may first determine the probability f^{o}(a^{(t)}) (e.g., 224a) to predict whether further attachment should be added to an atom at step t, a^{(t)}, or should stop at a^{(t)} per Equations 18 and 19:

$${f}^{o} ( {a}^{( t )} ) = σ ( {V}_{1}^{o} {a}^{( t )} + {V}_{2}^{o} {h}_{s} + {V}_{3}^{o} {h}_{p} ) \qquad ( Eq . 18 )$$

$$where$$

$${h}_{s} = {\sum }_{{a}_{i} ∈ {𝒢}_{s}} {a}_{i} \qquad ( Eq . 19 )$$

In Equation 18, a^{(t)} is the embedding of a^{(t)} calculated over the graph [symbol] *(t) (Equation 2); hₛ is the representation for all the synthons as in Eq. 19;

$${V}_{i}^{o} ’ s ( i = 1 , 2 , 3 )$$

are the learnable parameter matrices; σ is the sigmoid function. In Eq. 19, the synthon completion module 112 may calculate the representations by applying MPN over the graph [symbol] ₛ that could be disconnected, and the resulting representation is equivalent to applying MPN over each [symbol] ₛ's connected components independently and then summing over their representations. The synthon completion module 112 may intuitively measure “how likely” the atom has a new substructure attached to it by looking at the atom itself (i.e., a^{(t)},) all the synthons (i.e., hₛ), and the product (i.e., hₚ). In Eq. 18, BRICS fragment information (i.e., a′ as in Equation 6) is not used because the fragments for the substructures that will be attached to a^{(t)} are not available until the substructures are determined.

Attachment Type Prediction (AATP): If a^{(t)} is predicted to attach with a new substructure, the synthon completion module 112 may predict the type of the new substructure, with the probabilities of all the substructure types in the vocabulary [symbol] per Equation 20:

$${f}^{z} ( {a}^{( t )} ) = softmax ( {V}_{1}^{z} {a}^{( t )} + {V}_{2}^{z} {h}_{s} + {V}_{3}^{z} {h}_{p} ) \qquad ( Eq . 20 )$$

where

$${V}_{i}^{z} ’ s ( i = 1 , 2 , 3 )$$

are the learnable parameter matrices. Higher probability for a substructure type z indicates that z is more likely to be selected as z^{(t)}. The atoms a∈z^{(t)} in the attached substructure are stored for further attachment; that is, they, together with any newly added atoms along the iterative process, will become a^{(T+1)} (T=t+1, t+2, . . . ) in a depth-first order in the retrospectively reactant graphs. The synthon completion module 112 may stop the entire synthon completion process after all the atoms in reaction centers and the newly added atoms are predicted to have no more substructures attached.

To train the synthon completion module 112, a training system (not shown) may be employed that uses the teacher forcing strategy and attaches the ground-truth fragments instead of the prediction results to the intermediate molecules during training. The training system can learn the predictors f^{o}(⋅) (Eq. 18) as well as the below f^{z}(⋅) (Equation 20) by minimizing their cross-entropy losses [symbol] ^{o} and [symbol] ^{z} per Equation 21:

$${min}_{Φ} {ℒ}^{o} + {ℒ}^{z} \qquad ( Eq . 21 )$$

where Φ is the set of parameters.

### Example Method of Operation

FIG. 2A shows an example method 200 of operation of the retrosynthesis prediction system 100 of FIG. 1 to generate candidate reactants for a given target molecule in accordance with an illustrative embodiment. In the example shown in FIG. 2A, the exemplary retrosynthesis prediction system 100 can generate reactants from products in two operations (202, 204). In operation 202, the exemplary retrosynthesis prediction system 100 converts a target molecule into a set of intermediate molecular structures (synthons) by identifying, via a reaction center identification operation, the reaction centers 206 (shown as “BF-center” 206a, “BC-center” 206b, and “A-center” 206c) and their associated score (shown as s^{b}(bᵢⱼ), s^{c}(bᵢⱼ), and s^{a}(aᵢ), respectively) associated with the likelihood that the reaction centers is likely. The identification is performed two types of molecular graph representation learning 208 (message passing over molecular graphs shown as “GMPN” ( [symbol] ^{P}) 208a and message passing over fragment graphs are shown as “FMPN” ( [symbol] ^{B}) 208b). The use of both molecular graphs and fragment graphs allows reaction centers to be evaluated from that different information and encoding perspectives. As discussed in FIG. 1, the molecular learning that informs the parameters to be used for the reaction center scoring is also trained on diverse molecular graphs and fragment graphs, thus, extending the learning to these different information perspectives and encoding perspectives.

As discussed in relation to FIG. 1, the method 200 and the associated system may use message passing networks to learn the atom embeddings aᵢ and node embeddings nᵤ over the molecular graph and BRICS graph of product. The method can then use the learned node embeddings nᵤ from product P to predict the reaction centers and split the products into multiple synthons S={S₁, S₂}. The method can calculate the synthon embeddings from the atom embeddings aᵢ.

Method 200 can sample latent variables from the distributions derived from the synthon embeddings and complete the synthon with the latent variables and synthon embeddings. Method 200 can also perform conditional embedding (conditioned on synthons) and corresponding conditional sampling. Method 200 may directly derive the embeddings of leaving groups from synthons. Instead of decoding the latent embeddings into a whole reactant step by step, in some embodiments, the method 200 may directly decode the latent embeddings into a leaving group. Method 200 may then derive another leaving group embeddings from the synthons hₛ.

While reactants are not directly encoded, the reactants may be used in the node predictions and topology predictions. The method can use the reactants to learn and calculate the embeddings (e.g., use an auto-encoder over reactants and leverage its latent embedding space).

In the example shown in FIG. 2A, example reaction center predictions are shown as 210a, 210b, and 210c for the three reaction centers evaluation (the reaction center are shown as 212), which includes the atoms and the bonds that can be changed during the reaction in order to synthesize the product.

For BF-reaction centers (210a), the method 200 can account for synthetic reaction scenarios in which new bonds could induce the changes of neighbor bonds (BTCP) by determining the predictor f^{b}(b_{i/jk}) (214) for neighbor bond changes for each neighboring bond b_{i/jk} (shown as 214′). The method 200 can predict whether the charge of aᵢ remains unchanged in reactants by determining the predictor f^{c}(aᵢ) (216) for atom aᵢ (shown as 216′).

For BC-reaction centers (210b) and A-reaction centers (210c), the method 200 can also predict whether the charge of aᵢ remains unchanged in reactants by determining the predictor f^{c}(aᵢ) for atom aᵢ (shown as “ACP . . . ” for this example).

Following reaction center identification, selection (e.g., top-K selection), and production of synthons, the method 200 includes completing (204) the synthons into reactants by sequentially attaching 218 bonds or strings to synthons. As discussed in FIG. 1, the synthon completion model training informs the parameters (e.g., hₚ and hₛ 222) to be used for the attachment scoring or prediction are also trained on diverse molecular graphs and fragment graphs (shown as GMPN 220a and FMPN 220b).

The synthon completion process can iteratively be attached in a ranked manner from the library of candidate substructures without any chemical rules or templates imposed for the attachment (e.g., in a template-free manner). At each iteration (e.g., 218), the completion operation 204 can determine the predictions for attachment types (AATP) (shown as f^{o}(a^{(t)}) 224a) and predictions for attachment continuity (AACP) (shown as f^{z}(a^{(t)}) 224b). The completion process generates the resulting final set of top-N reactants 108 (shown as 108′).

According to the dataset, there exist some popular leaving groups that are used to complete synthons. Based on this observation, in addition to completing synthons by adding small fragments one by one to the synthons, the method 200 could also directly predict the leaving groups and attach the entire leaving groups to synthons.

### Example Pipeline Operation

FIG. 2B shows an example pipeline operation 250 that integrated the retrosynthesis operation (e.g., 200) with a molecular prediction 252. The molecular prediction 252 (shown as “MODOF” 252) can employ a deep generative model for molecule optimization via one fragment modification.

In the example shown in FIG. 2B, the MODOF system 252 may receive a compound or molecule 254 to which it (252) generates a set of top candidate molecules that serve as target molecules 104 (shown as 104′) for retrosynthesis. The retrosynthesis operation 200 may be performed to generate a set of candidate reactants 108 (shown as 108′″). In some embodiments, the retrosynthesis operation can be used as feedback to the compound or molecule generation, e.g., target molecules or top reactants may be scored and scoring used to direct the synthesizability of the candidate molecule generation.

MODOF (252) is a deep generative model that uses molecular graph and junction tree graph models for molecule optimization. The MODOF system (252) modifies a given molecule through the prediction of a single site of disconnection at the molecule and the removal and/or addition of fragments at that site.

In an exemplary implementation, the MODOF system modifies one fragment of a molecule at a time by generating molecular graphs using GMPN and generating node embeddings over corresponding junction tree models using TMPN for a pair of molecules: an input molecule and a target molecule. A disconnect site is predicted from the nodes of the junction tree model as applied to the molecular graph. Each neighboring node to the disconnect site identified in the junction tree model is then provided a related fragment when applied to the molecular graph, and the number of resulting fragments depends on the number of nodes identified in the first molecule. A first fragment is then removed from the input molecule and the remaining fragments are merged from a neighboring node into an intermediate molecular representation. New fragments are then attached sequentially until the representation of the target molecule is attained.

A pipeline of multiple identical MODOF systems can be implemented into a MODOF pipeline to modify an input molecule at multiple disconnection sites. When implemented into a pipeline, the MODOF pipeline can modify multiple fragments at different disconnection sites iteratively, which enables easier control over and intuitive deciphering of the intermediate modification steps and facilitates better interpretability of the entire modification process.

Other generative methods typically encode the entire molecular graphs and generate whole, new molecules from an empty or randomly selected structure. Unlike those methods, the MODOF system can learn from and encode the difference between molecules before and after optimization. The learning and generative processes are thus less complex and can retain major molecular scaffolds, thereby better approximating the in vitro chemical modification. Thus, the MODOF system may better inform and direct in vitro molecule optimization because it only encodes and decodes the fragment that needs modification and facilitates better modification performance.

Further description and example operation of the MODOF system may be found in Chen et al., “A deep generative model for molecule optimization via one fragment modification,” which is incorporated by reference herein.

The pipeline operation 250 may evaluate (256) the predicted reactions 108 to remove candidate reactions that are less desirable for synthesizability. The evaluation operation 256 may calculate and employ metrics such as molecular complexity, including the synthetic accessibility score [47] and fractal dimensions [48]. The measurements may be used to assess the synthesizability of the predicted target molecules (i.e., products). MODOF can modify a molecule structure into a new one. The new molecule structure may not exist so it need to be synthesized, and the retrosynthetic model can be used to synthesize the molecule that MODOF generates.

The predicted reactions may be additionally evaluated using existing forward reaction prediction methods [49-53], which can predict the products given the reactants. The predicted products may be compared with the given molecules.

The analysis of the predicted reactions may also be carried out through searches of existing databases, including Reaxys [2] and SciFinder scholar [3]. In cases where the exact structures of the reagents or target molecules are not present in these databases, structure similarity searches may be carried out using the basic chemical scaffold and the site of reactivity. While specific functional groups attached to that scaffold may influence the reactivity of adjacent sites via electronic or steric interactions, the proposed reactions are expected to match previously reported patterns of reactivity for that chemotype.

The predicted reactions can also be assessed through simulation of the reactions using software such as Gaussian [84] and AMBER [85] to evaluate the free energies during the reactions.

The components of the pipeline 250 may be performed individually in their own respective set of operations.

### Experimental Results and Examples

A study was conducted to develop and evaluate a retrosynthesis prediction operation (“G²Retro”) that, for a given target molecule (i.e., product), can identify a set of reactants that can be used to synthesize the target molecule through one synthetic reaction.

The G²Retro system was configured to predict reactions of given target molecules by predicting their reaction centers, and then completing the resulting synthons by attaching small substructures. Based on a comparison against twenty baseline methods over a benchmark dataset, the G²Retro system was observed to achieve state-of-the-art performance under many evaluated metrics. The G²Retro system was also observed to provide diverse predictions and unique reactions (to those generated in other studies) that were accessed to be possible by synthetic chemists.

Connected after the deep generative models that have been developed to optimize small molecule structures and properties [36, 37] for lead optimization, it was observed that the G²Retro system has great potential to generate synthetic reactions for in silico generated drug-like molecules, and thus can substantially benefit the drug development process.

The study considered the retrosynthesis prediction problem as two subproblems, following semi-template-based approaches. The first subproblem was to identify the reaction center from the target molecule. The study defined the reaction center as a single bond that is either newly formed or has the bond type changed or a single atom with a changed hydrogen count during the reaction. The study also incorporated into the reaction center the bonds neighboring the reaction centers that have type changes induced by the newly formed bond and the atoms with charge changes within the target molecule (e.g., using the BTCP and ACP operation described herein). The reaction centers broke the product molecules into synthons, which were defined as “hypothetical units within the target molecule that represent a potential starting reagent in the retrosynthesis of that target molecule” [28].

The second subproblem was to convert the synthons into reactants. The reactants were considered correct in the study if they were reported feasible in the benchmark data, or they were considered reasonable based on domain knowledge and expertise. The reactions that were included in benchmark data were referred to as ground truth. The study used one ground-truth reaction for each product in the benchmark data, though it is understood that numerous feasible reactions may exist for each product.

Materials—Data Preprocessing and Experimental Settings: The study used a benchmark dataset described in Yan et al. [12], referred to as the “USPTO-50K” dataset because it contains 50K chemical reactions that were randomly sampled from a large dataset collected by Lowe [29] from US patents published between 1976 and September 2016.

Each reaction in the large dataset was atom-mapped so that each atom in the product was uniquely mapped to an atom in the reactants. The 50K reactions in USPTO-50K were classified into 10 reaction types by Schneider et al. [30]. To avoid the information leakage issue [12](e.g., the reaction center is given in both the training and test data), all the product SMILES strings in USPTO-50K are canonicalized. The study used exactly the same training/validation/test data splits of USPTO-50K as in the previous methods [12, 17], which contain 40K/5K/5K reactions, respectively.

The study trained the G²Retro models on the 40K training data, with parameters tuned on the 5K validation data and tested on the 5K test data. Improving upon the prior work [4, 5, 7, 17], the study used the top-k (k=1, 3, 5, 10) accuracy to evaluate the overall performance of all the methods. Top-k accuracy is the ratio of test products that have their ground truth correctly predicted among their top-k predictions. Higher top-k accuracy indicates better performance. Also improving upon the prior work [7], the G²Retro system used the top-k accuracy (k=1, 2, 3, 5) to evaluate the performance of reaction center identification and synthon completion.

Baseline Methods. The study compared the G²Retro system with the state-of-the-art baseline methods for several one-step retrosynthesis problems, including five template-based (TB) methods, ten template-free (TF) methods and five semi-template-based (Semi-TB) methods. Table 9 shows performance in top-k accuracy of the G²Retro system compared to that of the evaluated state-of-the-art baseline methods.

TABLE 9

| Method | | Reaction type known | | | | Reaction type unknown | | | |
|---|---|---|---|---|---|---|---|---|---|
| type | Method | 1 | 3 | 5 | 10 | 1 | 3 | 5 | 10 |
| TB | Retrosim | 52.9 | 73.8 | 81.2 | 88.1 | 37.3 | 54.7 | 63.3 | 74.1 |
| | Neuralysm | 55.3 | 76.0 | 81.4 | 85.1 | 44.4 | 65.3 | 72.4 | 78.9 |
| | GLN | 64.2 | 79.1 | 85.2 | 90.0 | 52.5 | 69.0 | 75.6 | 83.7 |
| | MHNreact | — | — | — | — | 50.5 | 73.9 | 81.0 | 87.9 |
| | LocalRetro | 63.9 | 86.8 | 92.4 | 96.3 | 53.4 | 77.5 | 85.9 | 92.4 |
| TF | SCROP | 59.0 | 74.8 | 78.1 | 81.1 | 43.7 | 60.0 | 65.2 | 68.7 |
| | LV-Trans | — | — | — | — | 40.5 | 65.1 | 72.8 | 79.4 |
| | GET | 57.4 | 71.3 | 74.8 | 77.4 | 44.9 | 58.8 | 62.4 | 65.9 |
| | Chemformer | — | — | — | — | 54.3 | — | 62.3 | 63.0 |
| | Graph2SMILES | — | — | — | — | 51.2 | 66.3 | 70.4 | 73.9 |
| | TiedTransformer | — | — | — | — | 47.1 | 67.1 | 73.1 | 76.3 |
| | GTA | — | — | — | — | 51.1 | 67.6 | 74.8 | 81.6 |
| | Dual | 65.7 | 81.9 | 84.7 | 85.9 | 53.6 | 70.7 | 74.6 | 77.0 |
| | Retroformer | 64.0 | 82.5 | 86.7 | 90.2 | 53.2 | 71.1 | 76.6 | 82.1 |
| | MEGAN | 60.7 | 82.0 | 87.5 | 91.6 | 48.1 | 70.7 | 78.4 | 86.1 |
| Semi- | Retro Xpert | 62.1 | 75.8 | 78.5 | 80.9 | 50.4 | 61.1 | 62.3 | 63.4 |
| TB | G2G | 61.0 | 81.3 | 86.0 | 88.7 | 48.9 | 67.6 | 72.5 | 75.5 |
| | GraphRetro | 63.9 | 81.5 | 85.2 | 88.1 | 53.7 | 68.3 | 72.2 | 75.5 |
| | RetroPrime | 64.8 | 81.6 | 85.0 | 86.9 | 51.4 | 70.8 | 74.0 | 76.1 |
| | G²Retro | 63.1 | 84.2 | 88.5 | 91.7 | 53.9 | 74.6 | 80.7 | 86.6 |
| | G²Retro-B | 63.6 | 83.6 | 88.4 | 91.5 | 54.1 | 74.1 | 81.2 | 86.7 |

In Table 9, columns with “1”, “3,” “5,” and “10” indicate the “top-1,” “top-3,” “top-5” and “top-10” accuracies, respectively (in percentages). The best top-k accuracy values among the methods of each type are emphasized in bold. Table 9 was generated following the standard protocol in literature [4-7, 12, 17, 18, 21, 26, 27](1) when the reaction type was given a priori for both model training and inference (i.e., “Reaction type known”); and (2) when the reaction type was always unknown (i.e., “Reaction type unknown”). When the reaction type was known, the G²Retro system used a one-hot encoder as an additional feature for each atom in the product molecules indicating the reaction type.

The five template-based baseline methods [4, 17-20] first mine the reaction templates from training data and apply only these templates to construct reactants from the target molecule. Retrosim [17] selected the templates of reactions that produce molecules most similar to the target molecule. Neuralsym [18] predicted suitable templates using product fingerprints through a multi-layer perceptron. GLN [4] predicted reactions using two energy functions, one for template scoring and the other for reactant scoring conditioned on templates. MHNreact [19] learned the associations between molecules and reaction templates using modern Hopfield networks, and selected templates based on the associations. LocalRetro [20] selected templates against each atom and each bond using classifiers.

The ten templaGte-free baseline methods [6, 13-15, 21-23, 25, 27, 31] used Transformer over SMILES string representations of products and/or reactants. SCROP [13] mapped the SMILES strings of products to the SMILES strings of reactants using a Transformer, and then corrected the syntax errors (e.g., mismatch of parentheses in SMILES strings) to ensure valid reactant SMILES strings. LV-Trans [14] pre-trained a vanilla Transformer using reactions generated from templates and then fine-tuned the Transformer with a multinomial latent variable representing reaction types. GET [21] trained standard Transformer encoders and decoders using the combined atom representations learned from molecular graphs and from SMILES strings. Chemformer [22]translated product SMILES strings into reactant SMILES strings using Transformer, which is pre-trained on an independent dataset to recover masked SMILES strings (i.e., with some atoms masked out) or to normalize augmented SMILES strings (i.e., multiple, equivalent non-canonical SMILES strings for each SMILES string). Graph2SMILES [23] encoded molecular graphs using graph neural networks with attention mechanisms and decoded the reactant SMILES strings from the graph representations using a Transformer decoder. TiedTransformer [15] used two Transformers with shared parameters to learn the transformation from products to reactants and vice versa, respectively and selected the best reactions using the likelihood values from these two Transformers. GTA [31] enhanced a Transformer with truncated attention connections regulated by molecular graph structures. Dual [6] used an energy-based model with two Transformers to learn the transformation from product SMILES strings to reactants' SMILES strings and vice versa and selected the best reactions using the energy. Retroformer [25] integrated a reaction center detection module within a Transformer and decoded reactants utilizing the predicted reaction centers. MEGAN [27]transformed the product molecule graphs into the corresponding reactant graphs using a sequence of graph edits (e.g., change atom charges, add a new bond) that are learned from products and their reactants in the training set.

The semi-template-based methods [5, 7, 12, 26] may use molecular graph representations. Most of them explicitly predicted the reaction centers first. RetroPrime [26] trained two Transformers independently to predict the transformation from the product to its synthons and from the synthons to the reactants, respectively. RetroXpert [12] predicted reaction centers on molecular graphs via a graph attention network and transformed the resulting synthons to reactants using a Transformer. G2G [5] predicted the reaction centers on molecular graphs via a graph neural network and completed synthons into reactants through sequential additions of new atoms or bonds using the latent variables sampled from the latent space of a variational graph autoencoder. GraphRetro [7] predicted the reaction centers via a message passing neural network over molecular graphs and completed synthons by selecting the subgraphs in a vocabulary that realized the difference between the synthons and reactants.

Comparison with Semi-Template-Based (Semi-TB) Methods

When the reaction type is known, compared to other Semi-TB methods, the G²Retro system was observed to achieve the best performance on top-3 (84.2%), top-5 (88.5%), and top-10 (91.7%) accuracies, corresponding to 3.2%, 2.9% and 3.4% improvement over those from the best baselines (81.6% for RetroPrime on top-3, 86.0%, and 88.7% for G2G on top-5 and top-10) on these three metrics. For top-1 accuracy, the G²Retro-B system achieved the third-best performance (63.6%) compared to those of RetroPrime (64.8%) and GraphRetro (63.9%) on this metric. While the G²Retro system may underperform on certain metrics, it was observed to be overall significantly better than, e.g., RetroPrime, on all the other metrics: The G²Retro system outperformed the system of RetroPrime on top-3 accuracy at 3.2%, on top-5 accuracy at 4.1%, and on top-10 accuracy at 5.6%.

When the reaction type is unknown, a similar trend is observed: the G²Retro-B system was observed to outperform all the Semi-TB baseline methods on all the top accuracy metrics, with 0.7% improvement over the best baseline GraphRetro on top-1 accuracy, 225 4.7%, 9.7% and 13.9% improvement over the best baseline RetroPrime on top-3, top-5 and top-10 accuracies. The G²Retro system had a performance similar to that of G²Retro-B, with a better top-3 performance 74.6% that is 5.4% improvement from that of RetroPrime.

While it is well known in synthetic chemistry that there are several well-characterized reaction types (Table 10), these types often have distinct patterns in their reactions and reaction centers. Table 10 presents the data statistics for the performance of the different reaction types.

TABLE 10

| | Percentage | Reaction type known | | | | Reaction type unknown | | | |
|---|---|---|---|---|---|---|---|---|---|
| Type Name | (%) | 1 | 3 | 5 | 10 | 1 | 3 | 5 | 10 |
| heteroatom | 30.3 | 62.3 | 84.1 | 90.2 | 94.4 | 56.1 | 77.2 | 84.4 | 91.3 |
| alkylation and | | | | | | | | | |
| arylation | | | | | | | | | |
| acylation and | 23.8 | 76.1 | 93.9 | 96.7 | 97.6 | 67.0 | 87.3 | 92.3 | 95.4 |
| related | | | | | | | | | |
| processes | | | | | | | | | |
| deprotections | 16.5 | 58.3 | 87.2 | 91.5 | 93.9 | 51.8 | 76.5 | 82.7 | 87.9 |
| C—C bond | 11.3 | 48.1 | 68.1 | 75.7 | 82.4 | 37.2 | 56.6 | 67.9 | 75.7 |
| formation | | | | | | | | | |
| reductions | 9.2 | 72.5 | 87.9 | 91.8 | 95.0 | 52.7 | 69.8 | 78.1 | 84.6 |
| functional | 3.7 | 50.5 | 69.0 | 75.5 | 81.0 | 42.4 | 52.7 | 60.9 | 67.9 |
| group | | | | | | | | | |
| interconversion | | | | | | | | | |
| heterocycle | 1.8 | — | — | — | — | — | — | — | — |
| formation | | | | | | | | | |
| oxidations | 1.6 | 86.6 | 91.5 | 92.7 | 95.1 | 62.2 | 80.5 | 85.4 | 91.5 |
| protections | 1.4 | 85.3 | 89.7 | 89.7 | 89.7 | 48.5 | 67.6 | 85.3 | 86.8 |
| functional | 0.5 | 95.7 | 95.7 | 95.7 | 95.7 | 78.3 | 82.6 | 87.0 | 87.0 |
| group addition | | | | | | | | | |

For example, acylation reactions are very common approaches to creating amide and sulfonamide linkages. They are known for their efficiency and high yields, especially when they involve acyl/sulfonyl halides [32]. The improved performance with known reaction types integrated into retrosynthesis model training demonstrates that leveraging a priori reaction type information could benefit retrosynthesis prediction in general. However, in real applications, reaction types are typically not available in retrosynthesis when only the target molecule is presented. The superior performance of G²Retro and G²Retro-B in “reaction type unknown” condition demonstrates their great utility in real applications.

Comparison with strong baseline semi-template approaches: GraphRetro and RetroPrime. GraphRetro has good top-1 accuracies but much worse results on other top accuracy metrics. According to its authors [7], GraphRetro was observed to bias its beam search to the most possible reaction center. Thus, it may prioritize the most possible reactants from the most possible reaction center at the very top of its predictions. However, if the most possible reaction centers are not the ground truth, GraphRetro would miss the ground truth in its beam search, resulting in poor performance on other top accuracy metrics.

RetroPrime achieved the best top-1 accuracy with reaction type known. It used augmented SMILES strings (i.e., each product has multiple, equivalent, non-canonical SMILES strings) in training the two sequence-to-sequence transformers. It is likely that top results in RetroPrime correspond to the ground truth but in different, augmented SMILES strings, and thus high top-1 accuracy but low and similar other top accuracies.

Compared to these baselines, the G²Retro system was observed to achieve the best performance on all the top accuracy metrics (except on top-1 accuracy when reaction types are known). High top-k accuracies at all different k are desired as they indicate the holistically high-ranking positions of the ground truth in the predicted reactions, and thus the capability of models to recover knowledge from data. High top-k accuracies with k>1 may signify novel yet plausible reactions. This may be because high top-k (k>1) accuracy can imply that there might be a few reactions different from the ground truth but are very possible and thus are ranked on top. Such reactions may enable novel discoveries.

Comparison between G²Retro and G²Retro-B: The G²Retro-B system was observed to perform slightly better than the G²Retro system when the reaction types are unknown, but worse than G²Retro when the reaction types are known. The G²Retro-B system integrated synthetically accessible fragments in atom embeddings (see, e.g., Equation 6). When the reaction types are unknown, the fragment information can provide additional local contexts to atoms, which could facilitate better decisions on reaction center prediction and synthon completion. When the reaction types are known, atom embeddings can directly integrate the reaction type information in the G²Retro system, which may outweigh the contextual information provided by the fragments, and thus the G²Retro-B system may not achieve additional performance improvement from the G²Retro system.

Comparison with template-free (TF) methods: The G²Retro system and the G²Retro-B system was also observed to also demonstrate superior or competitive performance compared to the TF methods on all the top accuracies. With reaction types known, the G²Retro system was the best on top-3, top-5 top-10 accuracies compared to all the template-free methods; with reaction types unknown, the G²Retro-B system was observed to be the best on top-3, top-5 and top-10 accuracies, and was the second best one on top-1 accuracy. For example, the G²Retro system has a 4.9% better score than that of the best TF method on top-3 accuracy (i.e., Retroformer) with the reaction types unknown. Most TF methods such as Dual and Chemformer have the competitive performance on top-1 accuracy but relatively worse results on other top accuracy metrics. This may be attributed to the limited diversity generated with such beam search [33] via TF methods with SMILES representations, which may have led to the limited variation in their predicted results, and thus low and similar top-3, top-5, and top-10 accuracies. This lack of diversity and richness in the predictions, in addition to the lack of interpretability during the chemical sequence transformation process, could hinder the application of TF methods in retrosynthesis prediction. Compared to TF methods, the G²Retro system best imitated the reversed logic of synthetic reactions with two steps: reaction center identification and synthon completion, and overall, to achieve better performance.

MEGAN was observed to have good top-10 accuracies, but consistently much worse top-1 accuracies than all the other methods. MEGAN edited the product graph into reactant graphs in a sequential manner. The poor performance on top-1 accuracy indicates that MEGAN's sequential edits may not generalize well to test data; while MEGAN can still identify the ground-truth reactants, they are not predicted as the most likely.

Comparison with template-based (TB) methods. The G²Retro system and the G²Retro-B system was observed to achieve competitive performance with those from the TB methods. With reaction types known, the G²Retro system achieved either the second or the third on all the top accuracies; with reaction types unknown, the G²Retro-B system achieved the best performance on top-1 (54.1%), and either the second or the third on all the other top accuracies. For example, with reaction-type unknown, the G²Retro-B system was observed to provide the second best on top-3 accuracy, with 3.8% difference from the best performance of LocalRetro; the G²Retro-B system slightly underperformed the second-best baseline MHNreact on top-10 (86.7% compared to 87.9% from MHNreact) and outperformed MHNreact on all the other metrics.

LocalRetro is a very strong TB method. It can extract 731 templates from the benchmark training data, whereas other TB methods have much more templates (11,647 for GLN and 9,162 for MHNreact). Therefore, LocalRetro could achieve better template selection over a small template set compared to others over much larger template sets. However, LocalRetro may suffer from scalability issues on large datasets because it may score all the reaction templates on all the potential reaction centers (i.e., all atoms and all bonds) in the product molecules. In general, all TB methods may not generalize well to novel reactions that are not covered by the templates [7]. Unlike LocalRetro, the G²Retro system does not use reaction templates and only scores all the bonds and atoms once for reaction center identification, and thus is much more scalable in inference. It also can learn the patterns from training data and thus has a better chance to discover new patterns from the training data that are not covered by templates.

### Individual Module Performance Evaluation

Table 11 shows the performance of the two modules of the G²Retro system, namely the reaction center identification and the synthon completion.

TABLE 11

| | | Coverage | Reaction type known | | | | Reaction type unknown | | | |
|---|---|---|---|---|---|---|---|---|---|---|
| Module | Method | (%) | 1 | 3 | 3 | 5 | 1 | 2 | 3 | 5 |
| Reaction center | G2G | 97.9 | 90.2 | 94.5 | 94.9 | 95.0 | 75.8 | 83.9 | 85.3 | 85.6 |
| identification | | | (92.1) | (96.5) | (96.9) | (97.0) | (77.4) | (85.7) | (87.1) | (87.4) |
| | GraphRetro | 95.0 | 84.6 | 92.2 | 93.7 | 94.5 | 70.8 | 85.1 | 89.5 | 92.7 |
| | | | (89.1) | (97.1) | (98.6) | (99.5) | (74.5) | (89.6) | (94.2) | (97.6) |
| | RetroPrime | 100.0 | 84.6 | 94.0 | 96.7 | 97.9 | 65.6 | 81.3 | 87.7 | 92.0 |
| | | | (84.6) | (94.0) | (96.7) | (97.9) | (65.6) | (81.3) | (87.7) | (92.0) |
| | G²Retro | 97.5 | 84.3 | 94.6 | 96.5 | 97.0 | 69.5 | 85.6 | 90.8 | 94.8 |
| | | | (86.5) | (97.0) | (99.0) | (99.5) | (71.3) | (87.8) | (93.1) | (97.2) |
| | G²Retro-B | 97.5 | 85.0 | 94.1 | 96.2 | 97.3 | 69.3 | 85.4 | 91.1 | 94.7 |
| | | | (87.2) | (96.5) | (98.7) | (99.8) | (71.1) | (87.6) | (93.4) | (97.1) |
| Synthon | G2G | 100.0 | 66.8 | — | 87.2 | 91.5 | 61.1 | — | 81.5 | 86.7 |
| completion | GraphRetro | 99.7 | 77.4 | 89.5 | 94.2 | 97.6 | 75.6 | 87.4 | 92.5 | 96.1 |
| | | | (77.6) | (89.8) | (94.5) | (97.9) | (75.8) | (87.7) | (92.8) | (96.4) |
| | RetroPrime | 100.0 | 75.0 | — | 88.9 | 90.6 | 73.4 | — | 87.9 | 89.8 |
| | G²Retro | 100.0 | 72.8 | 85.6 | 90.2 | 93.0 | 73.3 | 84.6 | 89.6 | 92.8 |

Comparison of reaction center identification. Among all the Semi-TB methods, the definitions of reaction centers vary. In G2G, reaction centers are referred to as the only one newly formed bond during the reaction, and reaction center identification predicts whether there is such a new bond (and its location) or not in the products as in a classification problem. This reaction center definition and classification can cover 97.9% of the test data (the rest 2.1% correspond to multiple newly formed bonds). GraphRetro defines the reaction center as the newly formed bond (e.g., BF-center), the changed bond (e.g., BC-center), and the single atom with changed hydrogen count (e.g., A-center), which in total covers 95.0% of the reactions in the test set. RetroPrime aimed to identify all the atoms involved in the reactions as reaction centers, which covers all the reactions in the test set. The G²Retro system extended and improved the definition of the reaction center, e.g., in GraphRetro, to include induced bond type change and atom charge changes to cover 97.5% of the test set.

Due to the data leakage issue as revealed by Yan et al. [12](i.e., the reaction center is given in both the training and test data), the reported G2G reaction center identification performance, as cited in Table 11, may be overestimated [1]. GraphRetro used two functions, one for bonds and one for atoms, to predict reaction centers. While these functions were able to predict well when such bonds and atoms are truly reaction centers (i.e., performance in parentheses in Table 11), GraphRetro's reaction center definition covered the least (95%) of the test set compared to the other methods, resulting in still low accuracies (i.e., performance outside parentheses) over the test set. RetroPrime had a very generic definition of reaction centers—any atoms involved in the reactions and used one unified model to predict these atoms. However, as these atoms may experience different changes (e.g., connected to or disconnected from other atoms), a unified model not customized to specific changes may not suffice, leading to overall relatively low accuracies compared to other methods, particularly when reaction types are unknown.

The G²Retro and G²Retro-B systems were observed to have the most comprehensive definition of reaction centers (Section “Reaction Center Identification”) with high coverage (97.5%) on the test set. In addition, the G²Retro and G²Retro-B systems used a unique predictor for each of the reaction center types, which may contribute to the best overall accuracy among the entire test set, as well as good performance over the reactions covered by its reaction center definition.

Comparison of synthon completion: To compare synthon completion, all the reaction centers defined by different methods were given and used to start the completion processes. G2G predicted only bond establishment in its reaction center identification and thus had to deal with any associated changes, such as bond type change in its synthon completion process, which can complicate the synthon completion prediction. GraphRetro formulated the synthon completion as a classification problem over all the subgraphs that can realize the difference between the synthons and reactants. Its synthon completion may not guarantee to work for all possible products (e.g., 99.7% coverage over the test set) if the needed subgraph is not included in the pre-defined vocabulary. Among all the products that GraphRetro could handle, its synthon completion performance appeared to be the best, e.g., due to that classification can be much easier than a generation, as all the other methods do. However, since GraphRetro does not do well in reaction center identification, overall, it did not outperform other methods in retrosynthesis prediction, as Table 10 demonstrates.

RetroPrime transformed the synthons to reactants using a Transformer, but similarly to G2G, it also needed to deal with additional predictions, such as bond type change. RetroPrime's synthon completion performed reasonably well on top-1 accuracies. Together with its good top-1 accuracy on reaction center identification, RetroPrime achieved the best top-1 accuracy with reaction type known as demonstrated in Table 10. The G²Retro system does not use the BRICS fragments in synthon completion because the fragment information is not available for the substructures that will be attached to synthons.

Compared to GraphRetro, the G²Retro system leveraged a generative process to add substructures to synthons in synthon completion, which is inherently more difficult than classification as in GraphRetro. On average, the G²Retro system was observed to outperform RetroPrime, particularly on top-3 and top-5 accuracies. Combined with the strong performance on reaction center identification, the two modules together in the G²Retro system were observed to provide strong retrosynthesis prediction performance, e.g., as shown in Table 10.

Performance on Different Reaction Types: Table 9 shows the top-k accuracy (k=1, 3, 5, 10) of the reactions of different types. As shown in the table, The G²Retro processes appear to predict certain reaction types more accurately than others. This may be attributed to the relative structural diversity among potential reactants, particularly for substrates that can all provide the same products. For example, in the case of oxidations, only a very limited set of substrates can be utilized to generate a ketone, most commonly the oxidation of an alcohol, although ketones can be accessed through other types of reactions as well. This may lead to relatively higher accuracies of the G²Retro system for the reactions of oxidations (e.g., 62.2% top-1 accuracy with reaction type unknown). For reductions, however, numerous substrates may be utilized to generate an amine, including reductions of amides, nitro groups, and nitriles, to name a few. In addition, there are numerous methods to access the same amines through various structurally unique deprotection reactions. The number of methods available to access a specific functional group, therefore, may make it more difficult to accurately predict which method has been used for a specific molecule, leading to the lower accuracies on reactions of deprotections (e.g., 58.3% top-1 accuracy with reaction type known). This may be the case in carbon-carbon bond-forming reactions as well, which can be assembled in a number of ways from various substrates, potentially leading to a somewhat lower prediction success rate (e.g., 37.2% top-1 accuracy with reaction type unknown). In addition, in molecules containing more than one functional group, there may often be multiple ways in which that molecule can be assembled by targeting each individual functional group as the reaction center. Thus, multiple valid reaction pathways are often considered by synthetic chemists in order to most efficiently construct a molecule.

In the exemplary implementation of the G²Retro system, reactions involving a single newly formed bond or a single atom can be predicted. It is contemplated that reaction types involving multiple newly formed bonds, such as heterocycle formation reactions to construct new rings, can be included to augment the single bond or atom prediction.

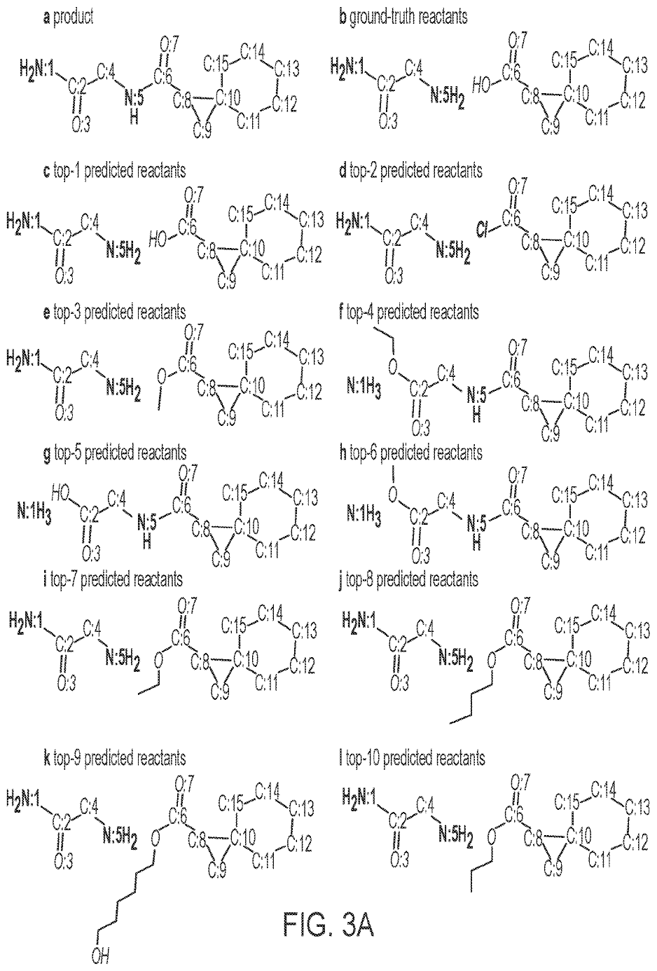

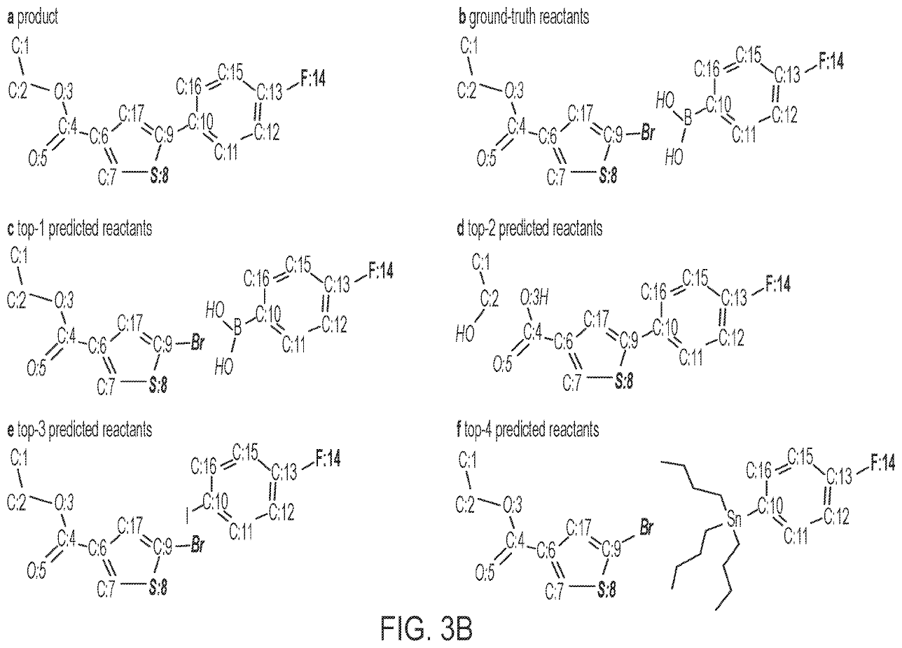

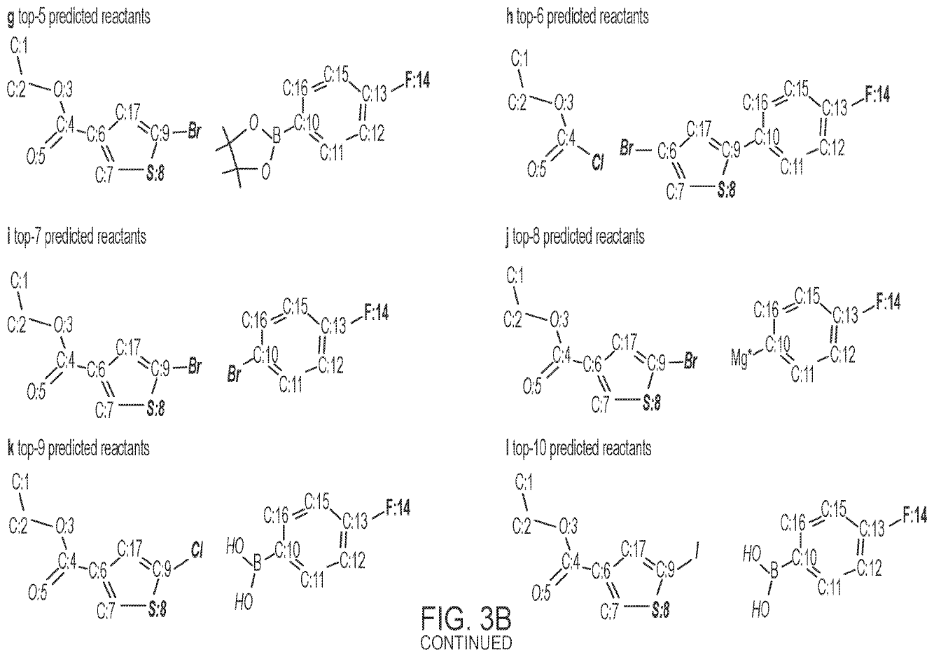

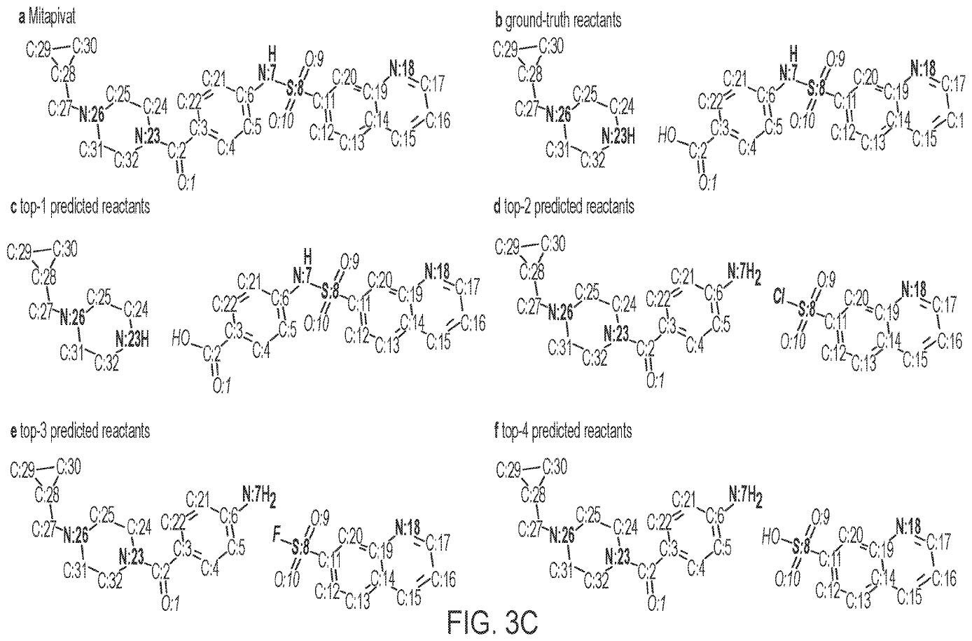

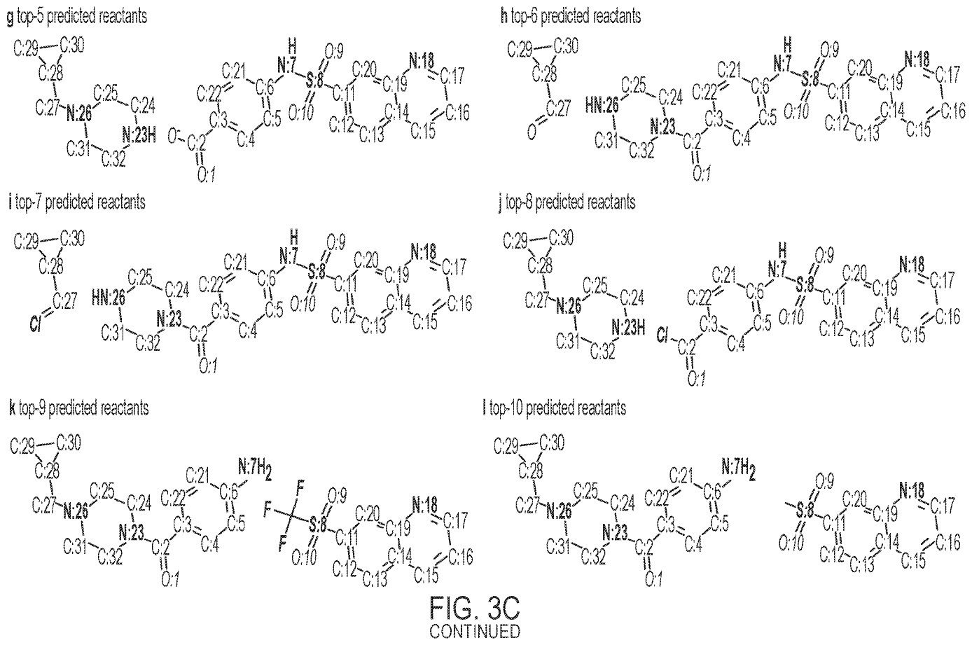

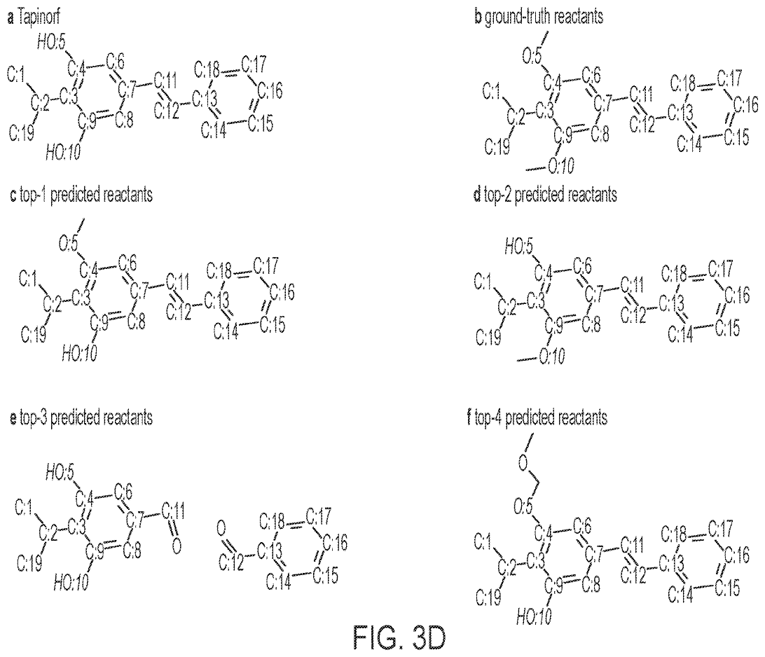

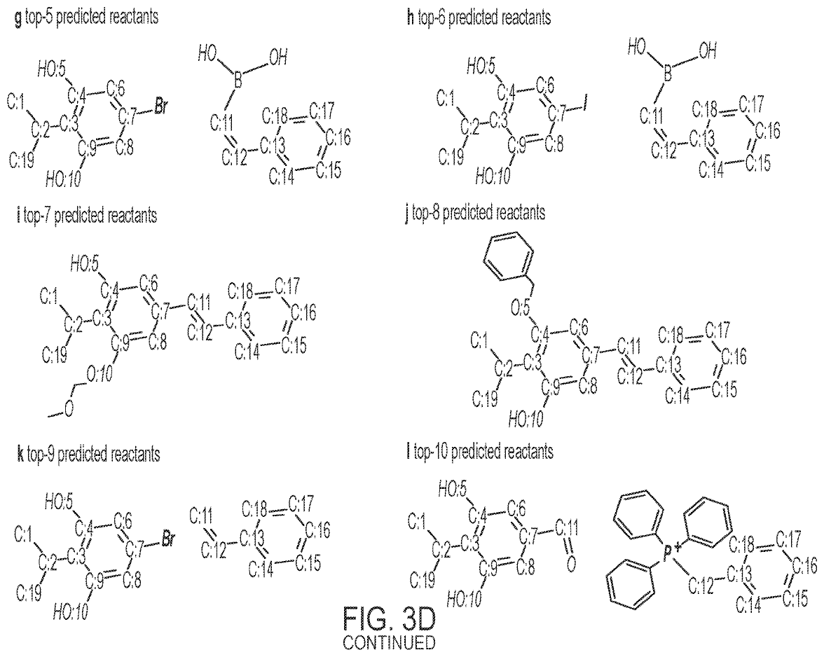

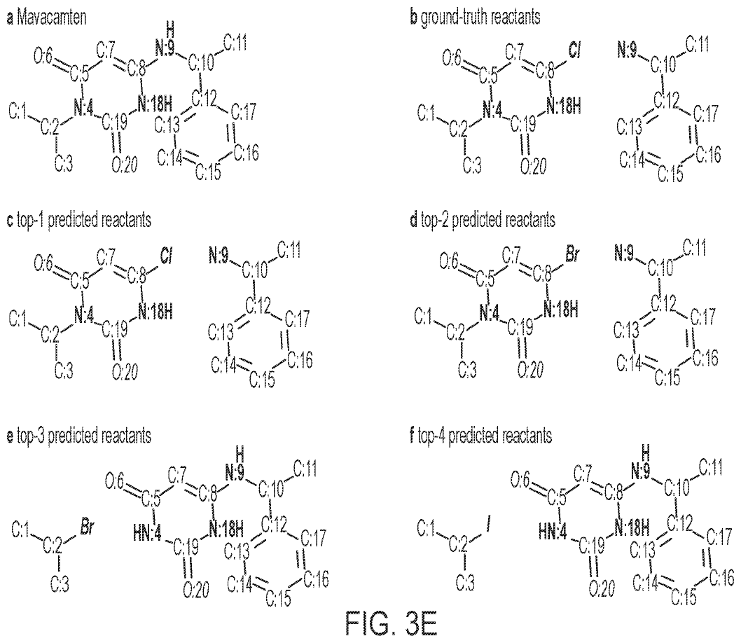

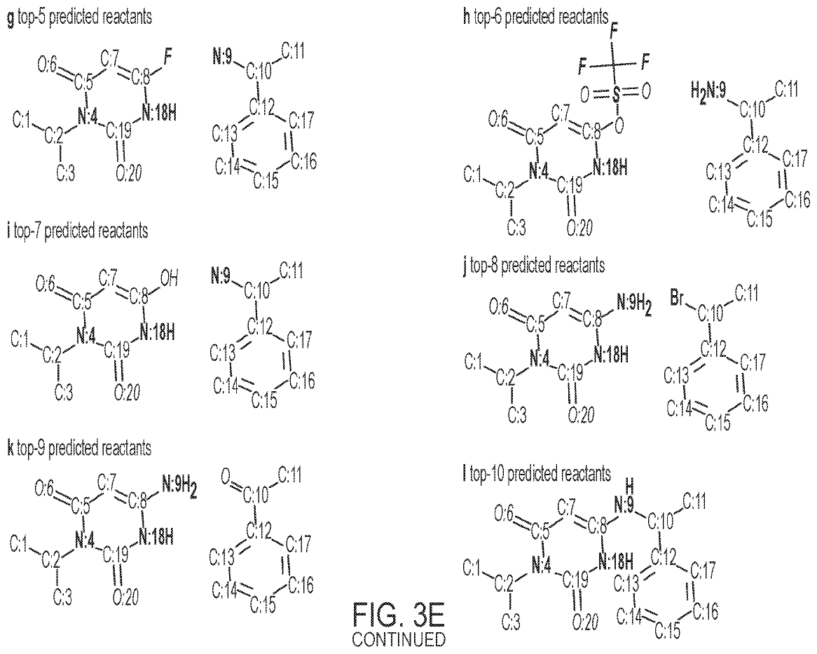

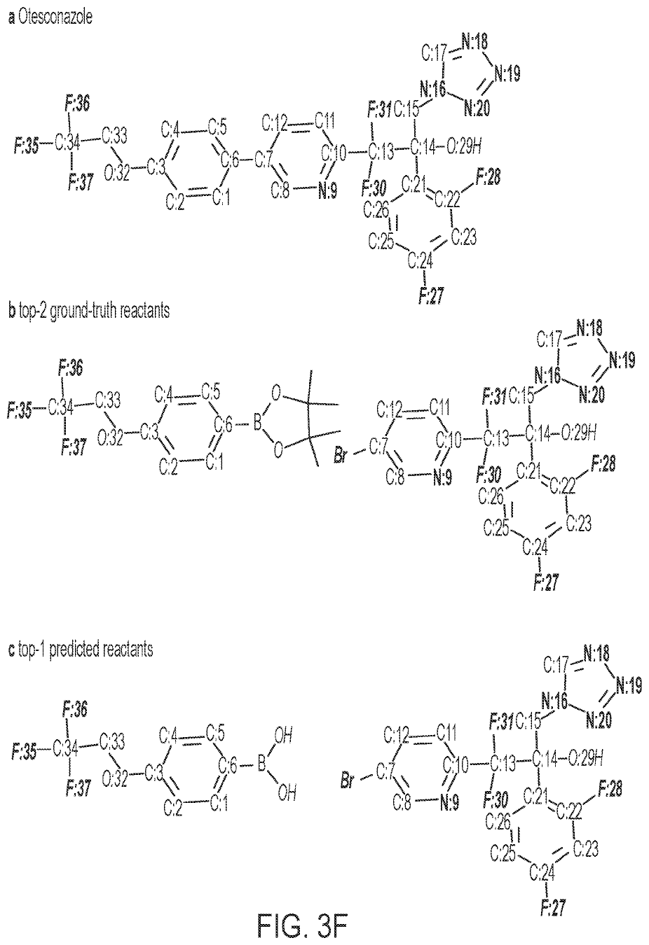

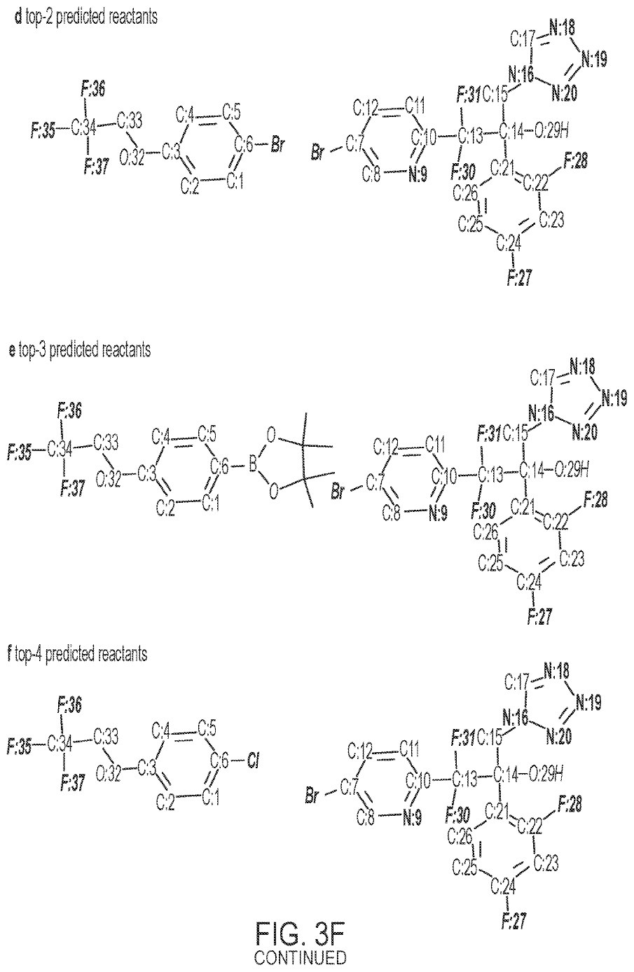

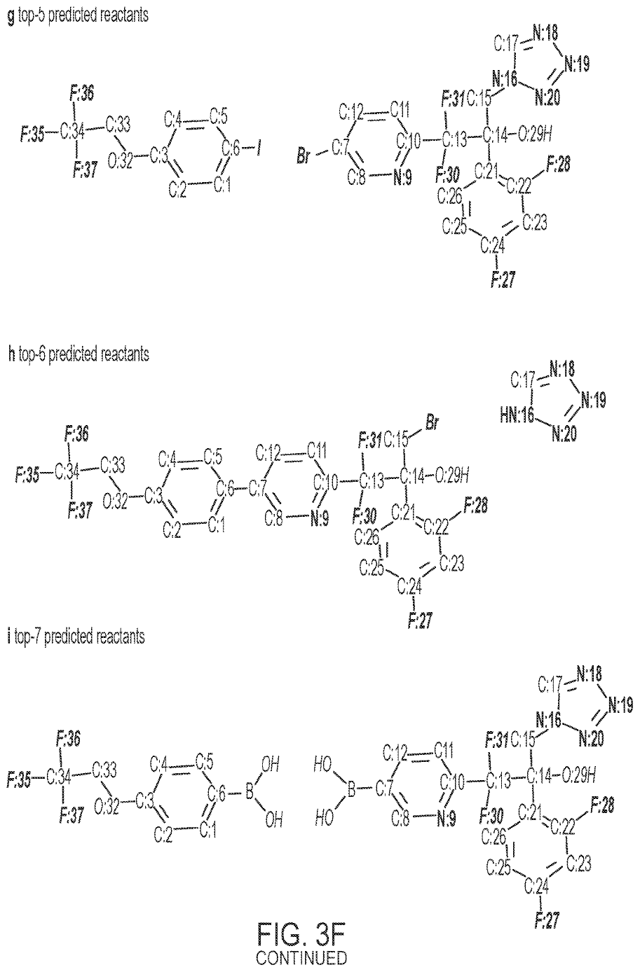

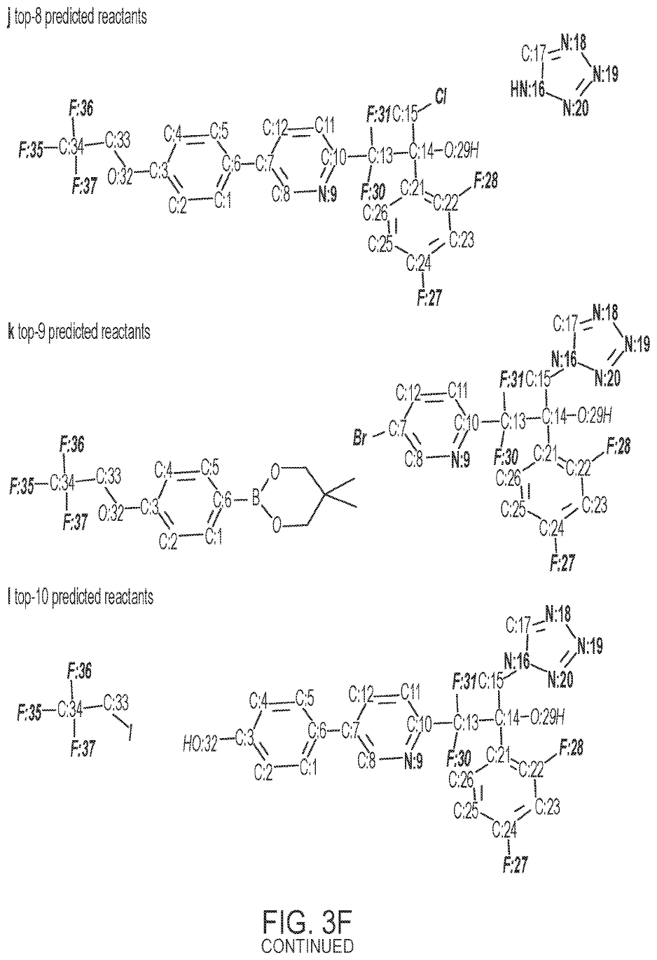

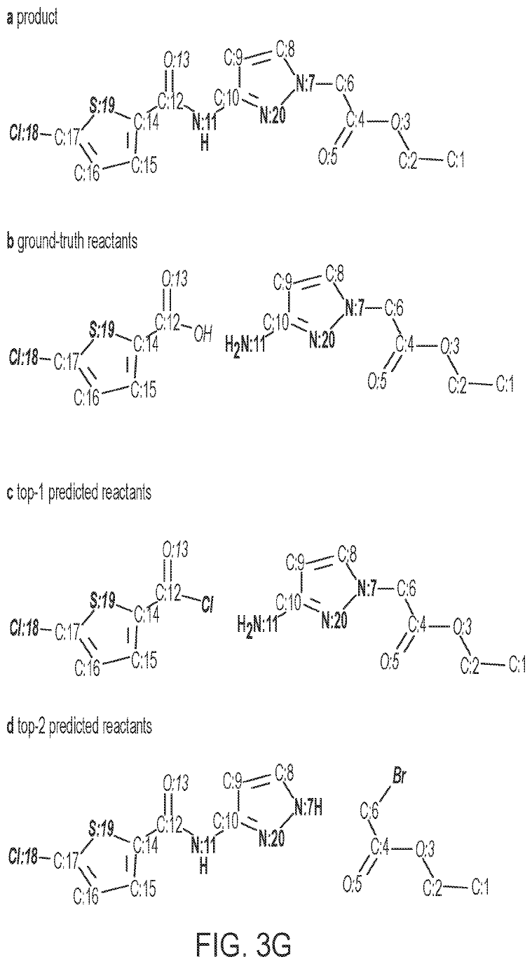

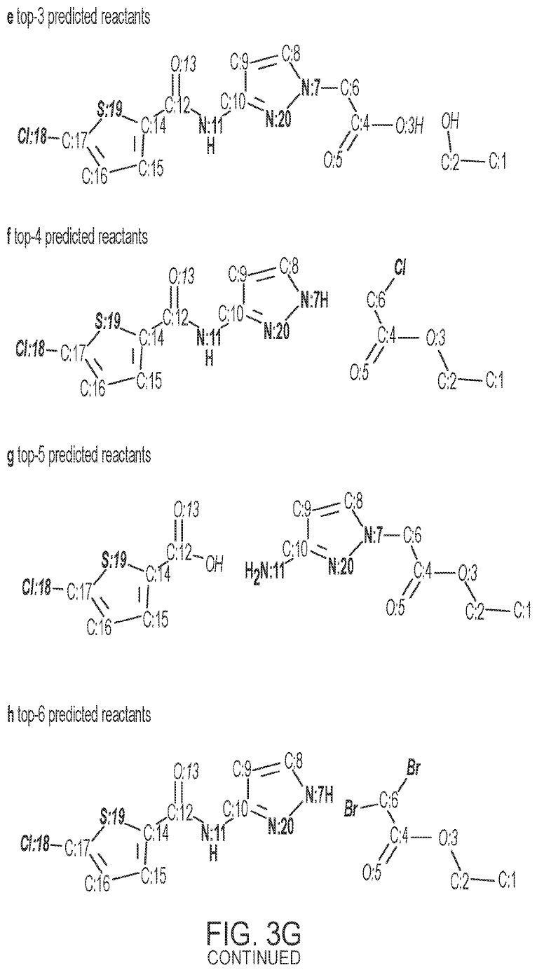

Case Studies: FIGS. 3A-3F each shows example outputs of the G²Retro system to illustrate the prediction of multiple reactions for each product due to multiple predicted reaction centers. This variability could be useful for chemical synthesis in order to consider all possible reaction strategies. In each of FIGS. 3A-3F, the top-10 predicted reactions are shown and discussed for their applicability and accuracy. In the example of FIG. 3A, the target product is NC(═O)CNC(═O)C1CC12CCCCC2. In the example of FIG. 3B, the target product is CCOC(═O)c1csc(−c2ccc(F)cc2)c1.

FIG. 3A, plot “a” (product) shows a target product containing amide linkages and was assembled (as described in the patent literature) by amide coupling reactions. FIG. 3A, plot “b” (ground truth reactants) shows an example ground truth used in the process.

FIG. 3A, plot “c” (top 1) shows an example output of the G²Retro system, which includes the ground truth of FIG. 3B, thus correctly predicting this coupling as a top-1 reaction for the construction of this molecule. The other predicted reactions are also instructive in informing the strengths of the G²Retro system.

In FIG. 3A, plot “a” (product), it can be observed that the product has two amide groups in the side chain of the molecule. It can be observed that the G²Retro system indeed can identify both of these linkages as potential reaction centers (e.g., in FIG. 3A, plot “c” a(top-1) between N:5 and C:6; in FIG. 3A, plot “g” (top-5) between N:1 and C:2). Typically, chemists would disconnect the molecule at the C:6 amide carbonyl rather than C:2 so that a fully elaborated side chain can be introduced to complete the molecule. This approach would generally be considered more efficient since its reaction introduces more complexity into the molecule in a single step and would therefore be predicted to limit the total number of steps necessary to construct the molecule. In certain cases, however, it may be necessary to introduce the nitrogen at N:1 last (e.g., shown in FIGS. 3A, plots “f” (top-4), “g” (top-5), and “h” (top-6)), so this should also be considered a feasible reaction.

In addition to the typical amide coupling strategy, which takes place between an amine and a carboxylic acid, the G²Retro system can also correctly identify the reaction of the amine with an acid chloride to make the same bond (FIG. 3A, plot “d” (top-2)). Although this was not the strategy utilized in the ground-truth study, the strategy is expected to work in this case for the construction of this molecule. Other common reaction that was predicted for this example was the nucleophilic addition of the N:5 (or N:1) amine into the C:6 (or C:2) carbonyl of an ester (N:5-C:6—FIG. 3B, plot “e” (top-3), “f” (top-4), “i” (top-7), “j” (top-8), “k” (top-9), “1” (top-10), and N:1-C:2—FIG. 3B, plots “f” (top-4) and “h” (top-6)). This type of reaction, which is essentially a transamidation reaction, should also work to provide the product.

In addition, G²Retro also predicted several different esters as substrates for this transformation (FIG. 3A, plots “c” (top-1), “e” (top-3), “i” (top-7), “j” (top-8), “k” (top-9), and “1” (top-10)). While these are different substrates, the variation of the ester side chain in these cases would not typically be considered significantly different by a synthetic chemist unless steric or electronic contributions affect the reactivity/electrophilicity of the ester carbonyl.

Retrosynthesis of the product in FIG. 3B involves a C—C bond-forming reaction between C:9 and C:10 (FIG. 3B, plot “a”). The disconnection of the carbon-carbon bond between the two aromatic rings, a heteroaromatic thiophene, and a benzene ring in this example, represents the most common disconnection in the molecule. In this example, the top-1 reaction (FIG. 3B, plot “c”) predicted by G²Retro for this transformation is a Suzuki coupling [34], a common metal-mediated coupling between a boronic acid reactant and a corresponding aryl halide. This common transformation is the same reaction observed in the ground truth (FIG. 3B, plot “b”).

In addition, the G²Retro system also identified additional permutations of this Suzuki reaction through the reversal of the reaction partners on the aromatic rings or changing the nature of the aryl halide (FIG. 3B, plots “k” (top-9) and “l” (top-10)). Traditionally, aryl chlorides (FIG. 3B, plot “k” (top-9)) are less reactive than aryl bromides or iodides (FIGS. 3B, plots “c” (top-1) and “1” (top-10)) for coupling reactions and in the past were considered unreactive in these reactions. Newer methods [35] using specially designed ligands, however, have made the use of such chlorides possible. The other difference observed in the predicted Suzuki couplings is the use of a boronic ester (FIG. 3B, plot “g” (top-5)) versus a boronic acid (FIG. 3B, plot “c” (top-1)). Both boronic acids and boronic esters are common reagents for these transformations, with many being readily available from commercial sources.

FIG. 3B, plot “d” (top-2) shows the G²Retro system also predicted that an esterification reaction at the C:4 carboxylic acid would also work to produce the product molecule. While it is potentially not as synthetically useful for building the molecule, it is a reasonable transformation.

In addition, the G²Retro system also predicted other coupling reactions [36] for the biaryl coupling reaction. These other methods include an Ullmann-type coupling [37](FIG. 3B, plot “e” (top-3) and “i” (top-7)), a Stille coupling [38](FIG. 3B, plot “f” (top-4)), and a Kumada coupling [35,39](FIG. 3B, plot “j” (top-8)).

In examples of FIGS. 3C-3F, the target products are clinically relevant drug molecules, Mitapivat, Tapinorf, Mavacamten, and Oteseconazole, respectively, in a showing of the predictive power of the G²Retro system.

FIG. 3C, plot “a” shows Mitapivat, a drug approved for hereditary hemolytic anemias [75]. The reported synthetic route of Mitapivat in the patent document [76] utilizes an amide coupling reaction to form the C:2-N:23 bond (FIG. 3C, plot “b” (ground-truth)). This is correctly predicted by G²Retro as the top-1 reaction (FIG. 3C, plot “c”).

As indicated by the top-5 reaction (FIG. 3C, plot “g”), G²Retro also predicts that the amide coupling reaction could be performed with the carboxylate salt of one of the reactants, a useful reactant under the right pH conditions. G²Retro also predicts that the acyl chloride as the substrate in this transformation would also react with the amine group and produce the desired molecule (FIG. 3C, plot “j” (top-8)).

In addition, G²Retro identifies the N:7-S:8 bond of sulfonamide linkage as the reaction center (e.g., FIG. 3C, plots “d” (top-2), “e” (top-3), “f” (top-4), “k” (top-9), “1” (top-10)). Most impressively, G²Retro predicts various S:8 sulfonyl groups reacting with the N:7 amine group, such as sulfonyl chloride (FIG. 3C, plot “d” (top-2)), sulfonyl fluoride (FIG. 3C, plot “e” (top-3)) and sulfonic acid (FIG. 3C, plot “f” (top-4)), which are theoretically feasible for the formation of the N:7-S:8 bond. G²Retro also predicts that the N:26-C:27 bond could be the reaction center and formed by the N:26 amine group reacting through a reductive amination with ketone in FIG. 3C, plot “h” (top-6) or through a nucleophilic substitution with the chloride in FIG. 3C, plot “i” (top-7).

In FIG. 3D, plot “a” (product) shows Tapinarof, a drug approved for plaque psoriasis and atopic dermatitis [79]. The reported synthesis in patent [80] constructs this drug by removing the protecting groups on 0:5 and 0:10 (FIG. 3D, plot “b” (ground-truth)). G²Retro correctly predicts the deprotection of the methyl groups on 0:5 (FIG. 3D, plot “c” (top-1)) or 0:10 (FIG. 3D, plot “d” (top-2)), which would work to produce the desired molecule, although the ground truth failed to be predicted due to the limitation of reaction centers. Similarly, G²Retro generates possible reactants that contain different types of protected alcohols, as seen with the methoxymethyl groups on 0:5 and 0:10 in FIG. 3D, plot “f” (top-4) and FIG. 3D, plot “i” (top-7) and the benzyl-protected 0:5 in FIG. 3D, plot “j” (top-8).

Most impressively, G²Retro also identifies the alkene linkage between C:11 and C:12 (FIG. 3D, plots “e” (top-3) and “1” (top-10)) and the C—C bond between C:7 and C:11 (FIG. 3D, plots “g” (top-5), “h” (top-6), “k” (top-9)) as reaction centers with various coupling reactions. These coupling reactions include McMurry coupling [81](FIG. 3D, plot “e” (top-3)), Wittig coupling [82](FIG. 3D, plot “1” (top-10)) and Suzuki coupling [34](FIG. 3D, plots “g” (top-5) and “h” (top-6)).

In FIG. 3E, plot “a” shows Mavacament, which was approved by FDA to treat hypertrophic cardiomyopathy [73]. The patent literature [74] reports the utilization of a nucleophilic aromatic substitution for the formation of the C:8-N:9 bond (FIG. 3E, plot “b” (ground-truth)). G²Retro correctly predicts this coupling as the top-1 reaction (FIG. 3E, plot “c”), and identifies its additional permutations by replacing the aryl chloride with the aryl bromide and the aryl fluoride, respectively (FIG. 3E, plots “d” (top-2) and “g” (top-5)). Aryl fluorides in FIG. 3E, plot “g” are not as typical as aryl chlorides and bromides, and G²Retro ranks the substitution reaction involving the aryl fluoride low.

In addition to the amine coupling strategy with aryl halides, G²Retro also identifies the reaction of the amine with trifluoro methyl sulfate to make the same bond (FIG. 3E, plot “h” (top-6)), which would be expected to work as with aryl halides in FIG. 3E, plots “c” (top-1) and “d” (top-2). However, the alcohol in FIG. 3E, plot “i” (top-7) is not a good enough leaving group to make the bond (i.e., C:8-N:9). Interestingly, G²Retro also identifies other amine linkages (e.g., in FIG. 3E, plot “e” (top-3) between C:2 and N:4; in FIG. 3E, plot “j” (top-8) between N:9 and C:10) as potential reaction centers. However, the proposed synthesis in FIG. 3E, plot “e” (top-3), and plot “f” (top-4) would most likely lead to the formation of undesired products as these reactant pairs would likely result in the alkylation of both N:4 and N:9. Therefore, the use of the aryl halides in FIG. 3E, plot “c” and plot “d” would be the more efficient way of obtaining the desired product.

In FIG. 3F, plot “a” shows Oteseconazole, a drug approved for recurrent vulvovaginal candidiasis [77]. In the patent literature [78], this drug is constructed by the C—C bond forming reaction between C:6 and C:7 and is assembled with Suzuki coupling [34] between an aryl bromide group and a boronic ester (FIG. 3F, plot “b” (ground-truth)). G²Retro correctly predicts this coupling as the top-1 with the boronic acid (FIG. 3F, plot “c”), the top-3, which is the same as the patented reaction (FIG. 3F, plot “e”), and the top-9 reaction with a relatively uncommon boronic ester (FIG. 3F, plot “k”).

Boronic acids in FIG. 3F, plot “c” would typically be considered by synthetic chemists as interchangeable with boronic esters, and thus should be considered a feasible reaction; while Boronic ester in FIG. 3F, plot “k” (top-9) should react in the same way with the patented reaction [78], and thus could deliver the desired compound. Interestingly, G²Retro also predicts the Ullmann-type coupling [37] with different aryl halides to construct the C:6-C:7 bond in FIG. 3F, plots “d” (top-2), “f” (top-4), and “g” (top-5), all of which would be expected as feasible reactions. G²Retro also identifies another C—N coupling of various aryl halides with imidazoles (C:15-N:16—FIG. 3F, plots “h” (top-6) and “j” (top-8)), which hypothetically would also work as expected. The results give confidence in the synthesizability of the G²Retro system in determining reactions or reactants for candidate compounds as, for example, generated via the MODOF system (e.g., 256).

The versatility predicted in the top-10 reactions may be of synthetic value for substrates if specific coupling methods fail or if the functionality necessary for one type of coupling reaction is not able to be easily prepared.

Diversity in predicted reactions: Diversity in predicted reactions is desired because of the potential to enable diverse discoveries on synthesis paths. The G²Retro system was observed to have the mechanisms to facilitate diverse predictions—that is, the beam search strategy in the G²Retro system appears to allow for multiple reaction centers and multiple different attachments and, therefore, potentially different scaffolds and structures in the predicted reactants. To analyze the diversity of G2Retro results, the study identified a set of products such that their third or fifth predicted reactions are the ground truth, referred to as having a hit at “3” or “5,” respectively. In this example, it was likely that their top-3 or top-5 predicted reactions were also possible.

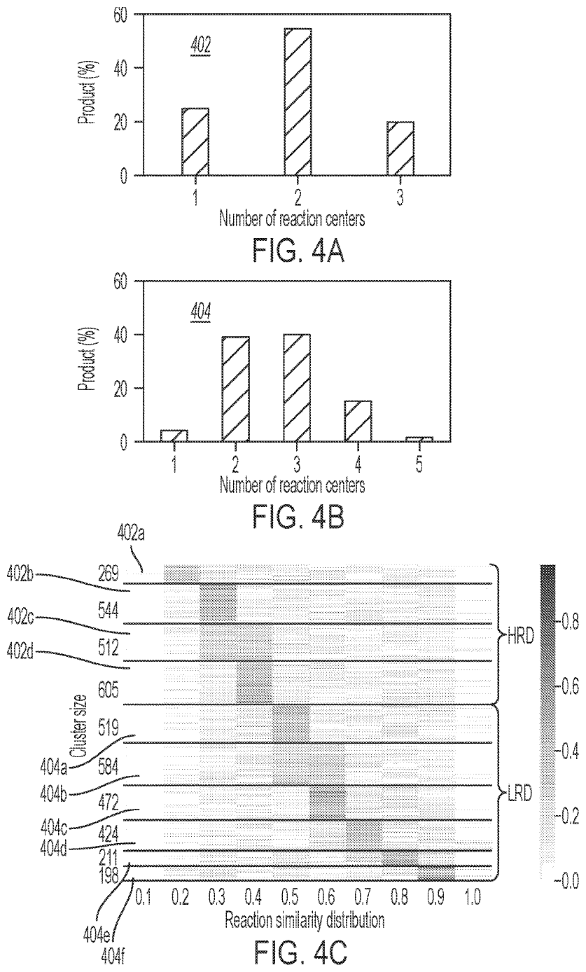

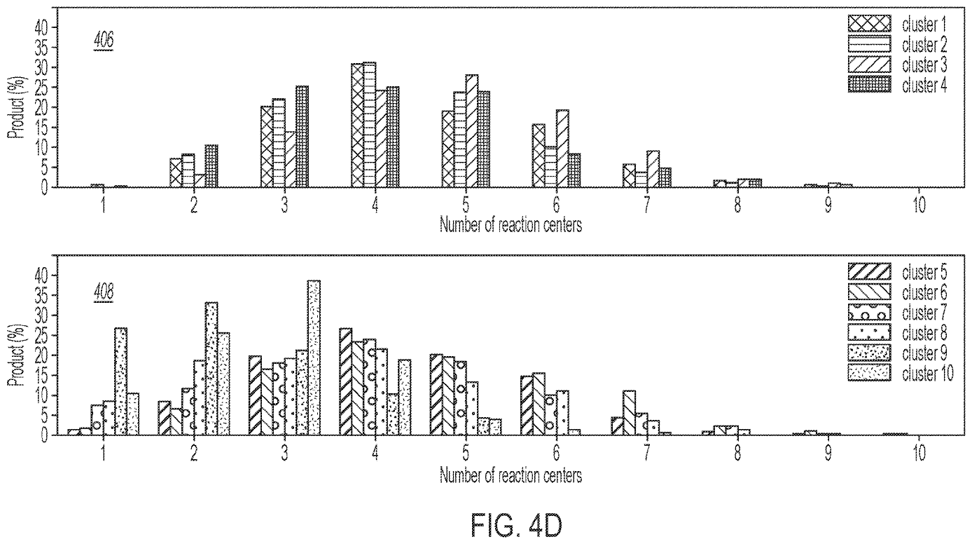

FIG. 4A shows the distributions of these products in terms of different reaction centers among their top-3 and top-5 predicted reactions. FIG. 4A shows that more than 50% of the products with a hit at “3” have their top-3 reactions from two different reaction centers; about 20% of the products have their top-3 reactions from three different reaction centers. FIG. 4B shows that for products with a hit at 5, almost 40% have two reaction centers, and another 40% have three reaction centers, among their top-5 predicted reactions; more than 10% have four reaction centers. Thus, FIGS. 4A and 4B clearly demonstrate the diversity in terms of different reaction centers in the G²Retro system predictions.

FIG. 3G presents an example of the diverse reactions predicted by the G²Retro system. For the product “CCOC(═O)Cn1ccc(NC(═O)c2ccc(C1)s2)n” (FIG. 3G), the G²Retro system predicted three reaction centers: an amide bond (between C:12 and N:11), a nitrogen-carbon bond (between N:7 and C:6) and ester and amide linkages (between C:6 and N:7). The patent (for this product molecule) reported that the target molecule was synthesized from a carboxylic acid derivative and an amine using amide coupling with a widely-used coupling reagent, EDC (FIG. 3G, plot “b” (ground-truth)).

The G²Retro system predicted an acyl chloride-amine reactant pair as the top-1 result (FIG. 3G, plot “c” (top-1)), a potentially viable and even high-yielding synthetic approach. It also predicted three reactant pairs from the other two reaction centers as possible routes within the top 4 (FIG. 3G, plot “d” (top-2) and “f” (top-4), which involves alkylation reactions to form the C:6-N:7 bond; FIG. 3G, plot “e” (top-3) at which forms the ester linkage between 0:3 and C:4).

To evaluate molecular similarities among the predicted reactants, the study calculated reaction similarities. For two possible reactions that result in the same product Mₚ, for example, R₁: M₁+M₂→Mₚ and R₂: M₃+M₄→Mₚ, the similarity between these two reactions may be determined per Equation 22:

$$sim ( {R}_{1} ; {R}_{2} ) = max ( s i {m}_{m} ( {M}_{1} ; {M}_{3} ) + s i {m}_{m} ( {M}_{2} ; {M}_{4} ) ; {sim}_{m} ( {M}_{1} ; {M}_{4} ) + s i {m}_{m} ( {M}_{2} ; {M}_{3} ) ) \qquad ( Eq . 22 )$$

where simₘ( ) is a similarity function over molecules. The study used the Tanimoto coefficient over 2,048-bit Morgan fingerprints as simₘ( ). For each product, the study calculated its all pairwise reaction similarities among all its top-10 reactions and used the distribution of the reaction similarities to measure reaction diversity; that is, lower reaction similarities indicate higher reaction diversity. The study clustered the products according to their reaction similarity distributions using the K-means clustering algorithm in Euclidean distances. Table 12 shows an exemplary algorithm implementation of clustering products according to reaction similarity distributions.

TABLE 12
Algorithm A8 Clustering products according to reaction similarity
distributions

$Require : {\{ {M}_{p}^{k} \cdot {\{ {R}_{i}^{k} \}}_{i = 1 , … , 10} \}}_{k = 1 , … , K} , NClusters$

| | |
|---|---|
| 1: | $for each {M}_{p}^{k} and {\{ {R}_{i}^{k} \}}_{i = 1 , … , 10} do$ |
| | [symbol] calculate pair-wise similarities among top-10 predictions |
| | (Equation 1) |
| 2: | $for each pair of reactions ( {R}_{i}^{k} , {R}_{j}^{k} ) with i \neq j do$ |
| 3: | ${s}_{ij}^{k} = sim ( {R}_{i}^{k} , {R}_{j}^{k} )$ |
| 4: | end for |
| | $⊳ generate the reaction similarity distribution of product {M}_{p}^{k}$ |
| 5: | ${h}^{k} = histogram ( {\{ {s}_{ij}^{k} \}}_{∀ i , j} )$ |
| 6: | end for |
| | [symbol] cluster products using K-Means according to their reaction |
| | similarity distributions |
| 7: | {Cᵢ}_{i=1,...,NClusters} = K-Means({h^{k}}_{k=1,...,K}, NClusters) |
| 8: | return {Cᵢ}_{i=1,...,NClusters} |

FIG. 4C shows the clustering results. As shown in FIG. 4C, the first four clusters (402a-402d) have on average lower reaction similarities (on average 0.46 among the four clusters; 0.41, 0.45, 0.45, 0.49 in each of the clusters, respectively), and thus are referred to as high-reaction-diversity clusters (HRD). The other six clusters (404a-404f), referred to as low-reaction-diversity clusters (LRD), have relatively higher reaction similarities (on average 0.58 for among the six clusters; 0.52, 0.53, 0.58, 0.62, 0.67, 0.67 in each of the clusters, respectively).

FIG. 4D shows the distributions of reaction centers of the two clusters from FIG. 4C. Comparing FIG. 4D, plot “a” (406) and FIG. 4D, plot “b” (408), HRD clusters tend to have more reaction centers than those in LRD, and the number of reaction centers correlates well with reaction diversity (−0.8486 between the average similarities and the number of reaction centers). Particularly, the first cluster, which has the highest reaction diversity (lowest reaction similarity), has, on average, 4.41 reaction centers, compared to the average 3.92 reaction centers of those in LRD clusters. The ninth and tenth clusters, which have the lowest reaction diversity, have on average 2.57 reaction centers. These results clearly show the diversity of the G²Retro system predictions.

### Discussion

Comparison fairness among existing methods. In the instant study of the baseline methods, several issues were identified that appear to introduce biases in method evaluation and impede a fair comparison among many existing methods. In Table 10, the RetroXpert's results were from its updated GitHub [40], as the results originally reported in the manuscript had a data leakage issue (all the reaction centers were implicitly given) and thus were overestimated [12]. G2G may also suffer from the data leakage issue, as discussed [41], but G2G's results were only available from its original paper, though likely overestimated. All the methods except Neuralsym, LV-Trans, Dual, and Retroformer published their code and datasets. Among these methods, most template-free methods, including SCROP, GET, Chemformer, TiedTransformer, GTA, and AT used the same data split, which is, however, different from the benchmark data split used in the other methods. For example, the training set of these template-free methods had 40,029 reactions, while the training set of the other methods, including the G²Retro study, had 40,008 reactions. Even though all the methods adopted the same ratio (i.e., 80%/10%/10% for training/validation/test set) to split the benchmark dataset, their splits, particularly their test sets, were not identical, leading to a potential unfair comparison among these methods. In the instant study, the study adopted the data split used by the previous semi-template-based methods; for the template-free methods with different data splits, the study used the results reported by their authors.

Comparison among template-based, template-free and semi-template-based methods. Template-based methods were first developed for retrosynthesis prediction. They match products into pre-defined templates that are extracted from training data or hand-crafted based on knowledge. A notable advantage of templates is that they can enable strong interpretability (e.g., each template may correspond to a certain reaction type, a chemical scaffold, or a reactivity pattern) and thus result in reactions that conform more to domain knowledge. They can also well fit the data if the templates are extracted from the data. However, they suffer from a lack of strong learning capabilities and a lack of generalizability if the templates do not cover and cannot automatically discover novel reaction patterns. In general, template-based methods underperform template-free and semi-template-based methods. Template-free methods largely leverage the technological advancement in Natural Language Processing (NLP), including large-scale language models such as Transformer and BART, and also many pre-training techniques. They formulate a reaction as a SMILES string translation problem. Rather than enumerating pre-defined patterns (i.e., templates) as template-based methods do, template-free methods are equipped with much stronger learning capabilities from SMILES strings and can represent latent reaction transformation patterns in an operable manner. Although with improved performance compared to that of the template-based methods, unfortunately, template-free methods sacrifice their interpretability as it is non-retrieval to decipher why an atom (analogous to a token in NLP) is generated next along the SMILES strings or what chemical knowledge the actions correspond to. In addition, as SMILES strings are a ‘flattened’ representation of molecular graphs according to the atom orderings from a graph traversal, template-free methods over SMILES strings cannot fully leverage molecular structures, which ultimately determine molecule synthesizability and reaction types.

Semi-template-based methods, typically over molecular graphs, represent the most recent and also, in general, the best performing retrosynthesis prediction methods. They utilize the most advanced graph representation learning paradigm to better capture molecule structures. They also take advantage of the graph (variational) auto-encoder frameworks or sequential predictions to empower the models with generative ability. More importantly, semi-template-based methods have the mechanism to enable diversity among predicted reactions by allowing multiple samplings from the latent space. Meanwhile, semi-template-based methods have two steps: (1) reaction center identification and (2) synthon completion, better complying with how chemical reactions are understood. G²Retro is a semi-template-based method and achieves superior performance to other methods, demonstrating it as a state-of-the-art method for retrosynthesis prediction.

In vitro validation. The use of top-k accuracy as the evaluation metric has been dominating in the current retrosynthesis prediction research. However, as we have demonstrated in our case study, top-k accuracy has serious limitations and underestimates model performance. It only compares the predicted reactions with those in the benchmark data but does not consider novel predicted reactions that are not in the benchmark data but are highly likely. Such novel predictions should be assessed using existing data from very large reaction databases and evaluated from the perspective of a synthetic chemist, so to determine the accuracy and likelihood that these predicted approaches could be employed. Finally, the predicted reactions should be prioritized for synthesis and executed in the laboratory to determine whether or not they proceed as predicted. Such in vitro testing and validation are very much needed ultimately to truly translate the computational approaches into real impacts. While no standard protocols for large-scale reaction validation exist, a funnel-shaped filtering protocol could be useful.

First, high-throughput prioritization of the predicted reactions should be conducted to identify a feasible set of reactions for further validation, as the retrosynthesis prediction methods can easily produce many reactions, not feasible for manual investigation or selection. Forward synthesis prediction methods [42, 43](i.e., predict products given reactants) trained from an independent dataset can be leveraged for quick prioritization. Such methods have been demonstrated to achieve good performance. A literature search (e.g., via SciFinder [3] or Reaxys [2]) could follow to identify from the prioritized set those predicted reactions that match previously reported patterns of reactivity. Domain expertise in the area of chemical synthesis will be critical to selecting reactions based on the literature support and the commercial availability of their starting materials for a small-scale in vitro validation. All of the above represent challenging but interesting future research directions.

Additional Discussion: Drug development is time-consuming and costly: it takes approximately 10-15 years and $1 to $1.6 billion1 to fully develop a new drug. One of the key steps in drug development is the identification of drug-like small molecules that display desired properties against a specific biomolecular target and then the synthesis of such molecules if they do not exist. Retrosynthesis is a procedure where such a desired molecule is transformed into potential reactants, and thus the synthesis routes are identified. An extensive, diverse library of high-quality synthesis paths for a given molecule can enable more feasible reaction solutions starting from commercially available chemical building blocks and provide more options for operationally simple, high-yielding transformations using widely accessible reagents. The success and efficiency of retrosynthesis of drug-like small molecules immensely impact the entire drug development process and affect its success rate, costs, and speed.

Current retrosynthesis analysis is primarily conducted by synthetic and medicinal chemists based on their knowledge and experience, which could be limited or susceptible to human error. Consequently, the planned synthetic routes may not be diverse enough to cover novel, economic or green reactions. There exist proprietary synthesis reaction databases manually curated from the literature, including Reaxys [2] and SciFinder [3]. Unfortunately, the high prices of these databases act to limit their accessibility in some academic and small biotech settings. Even with the aid of these databases, the development of new reactions and synthetic pathways for the preparation of challenging molecules remains non-trivial. In addition, database searches can be time-consuming with low throughput, particularly without extensive domain knowledge to guide the process. Recent in silico retrosynthesis prediction methods using deep learning [4-7] have enabled alternative computationally generative processes to accelerate the conventional paradigm. These deep-learning methods learn from string-based representations (SMILES) or graph representations of given molecules and generate possible reactant structures that can be used to synthesize these molecules, leveraging the advancement of natural language processing [8], graph neural networks [9], auto-encoders [10] and other techniques in deep learning. They have demonstrated strong potential to significantly accelerate and advance retrosynthesis analysis. The instant study focused on the one-step retrosynthesis prediction, which can predict the possible direct reactants for the synthesis of the target molecules and act as the foundation of multi-step retrosynthesis analysis [11].

Related work. Deep-learning-based retrosynthesis prediction methods are typically categorized into three classes: template based (TB), template free (TF), and semi-template based (Semi-TB).

Template-based (TB) methods. Template-based methods formulate the retrosynthesis problem as a selection problem over a set of reaction templates. These templates can be either hand-crafted by experts [16] or automatically extracted from known reactions in databases [4, 17-20]. Szymkuc et al. [16] provided a review on using reaction templates coded by human experts for synthetic planning. However, these rules may not cover a large set of reactions due to the limitation of human annotation capacity. Recent template-based methods extract reaction templates automatically from databases. With the reaction templates available, Coley et al. [17](Retrosim) selected the reaction templates that the corresponding reactions in the database have the products most similar with the target molecules to synthesize the target molecules. Dai et al. [4] learned the joint probabilities of templates matched in the product molecules and all its possible reactants using two energy functions, one for reaction template scoring and the other for reactant scoring conditioned on templates. Seidl et al. [19](MHNreact) learned to associate the target molecule with the relevant reaction templates using a modern Hopfield network. Chen et al. [20](LocalRetro) scored the suitability of all the reaction templates at all the potential reaction centers (atoms and bonds) in the target molecule. The use of templates provides interpretability towards the reasoning behind the generated reactions. However, these templates also limit the template-based methods to the reactions only covered by the templates.

Template-free (TF) methods. Template-free methods directly learn to transform the product into the reactants without using the reaction templates [6, 13-15, 21-25]. Most template-free methods utilize the sequence representations of molecules (SMILES) and formulate the transformation between the product and its corresponding reactants as a sequence-to-sequence problem. Many SMILES-based methods use Transformer [8], a language model with attention mechanisms to model the relationship across tokens. Transformer follows the encoder-decoder architecture, which encodes the product SMILES string into a latent vector and then decodes the vector into the reactant SMILES strings. For example, Tetko et al. [24](AT) learned to transform a product into its reactants using a Transformer trained on a dataset augmented with various non-canonical SMILES representations of each molecule. In AT, each target molecule was tested multiple times using different SMILES string representations. Kim et al. [15](TiedTransformer) learned the transformation from a product to its reactants using two coupled Transformers with shared parameters, one for the forward product prediction (synthesis) and the other for the backward reactant prediction (retrosynthesis). During the inference, they leveraged both the forward and backward models to find the best reactions. Sun et al. [6](Dual) transformed a product to its reactants using an energy-based framework. They also leveraged the duality of the forward and backward models by training them together and selecting the best reactions with the highest energy value from the two models. Template-free methods are independent of reaction templates and thus have better generalizability to unknown reactions. However, template-free methods lack interpretability toward the reasoning behind their end-to-end predictions. SMILES-based template-free methods also suffer from the validity issue that the generated sequences may fail to follow the grammar of SMILES strings or violate chemical rules [13].

Semi-template-based methods. Semi-template-based methods [5, 7, 12, 26, 27] do not use reaction templates, or they do not directly transform a product into its reactants. Instead, most semi-template-based methods follow a two-step workflow utilizing atom-mappings: (1) identify the reaction centers and transform the product into synthons (intermediate molecules) using the reaction centers; (2) complete the synthons into the reactants. Shi et al. [5](G2G) first predicted reaction centers as bonds that can be used to split the product into the synthons and then utilized a variational autoencoder to complete synthons into reactants by sequentially adding new bonds or new atoms. Somnath et al. [7](GraphRetro) predicted the bonds with changed bond types or the atoms with changed hydrogen count as the reaction centers and then completed the synthons by selecting the pre-extracted subgraphs that realize the difference between synthons and reactants. Wang et al. [26](RetroPrime) formulated the reaction center identification and synthon completion problems as two sequence-to-sequence problems (i.e., product to synthon and synthon to reactant) and trained two Transformers for these problems, respectively. The prediction of reaction centers first in the above methods allows better interpretability of the reasoning behind the generation process. The two-step workflow also empowers these methods to diversify their generated reactants by allowing multiple different reaction center predictions forwarded into their synthon completion step. Other semi-templated-based methods utilize the atom-mappings in a different way. For example, Sacha et al. [27](MEGAN) formulated retrosynthesis as a graph editing process from a product to its reactants. These graph edits include the change in the atom properties or the bond types or the addition of the new atoms or the benzene rings into the synthons.

The G²Retro system also identifies the reaction centers and then completes the synthons into the reactants in a sequential way, as G2G does. However, the G²Retro system is fundamentally different from G2G as the G²Retro system can cover multiple types of reaction centers while G2G takes only the newly formed bonds as the reaction center, which leads to lower coverage of G2G on the dataset. During synthon completion, the G²Retro system can attach substructures (e.g., rings and bonds) instead of single atoms as in G2G into synthons to simplify the completion process. In addition, the G²Retro system uses other synthons to complete a synthon, while G2G does not consider other systems. MEGAN also applies an action to add the benzene rings in synthon completion, but it cannot attach other complex ring substructures as performed by the G²Retro system. In addition, MEGAN does not follow the two-step workflow, and thus the G²Retro system is also fundamentally different from MEGAN.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

It must also be noted that, as used in the specification and the appended claims, the singular forms “a,” “an,” and “the” include plural referents unless the context clearly dictates otherwise. Ranges may be expressed herein as from “about” or “approximately” one particular value and/or to “about” or “approximately” another particular value. When such a range is expressed, other exemplary embodiments include from the one particular value and/or the other particular value.

By “comprising” or “containing” or “including” is meant that at least the name compound, element, particle, or method step is present in the composition or article or method, but does not exclude the presence of other compounds, materials, particles, method steps, even if the other such compounds, material, particles, method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, 4.24, and 5).

Similarly, numerical ranges recited herein by endpoints include subranges subsumed within that range (e.g., 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

The following patents, applications, and publications, as listed below and throughout this document, are hereby incorporated by reference in their entirety herein.
[1] Wouters, O. J., McKee, M. & Luyten, J. Estimated research and development investment needed to bring a new medicine to market, 2009-2018. JAMA 323, 844 (2020).
[2] Reaxys. Reaxys is a registered trademark of relx intellectual properties as used under license. Https://www.reaxys.com. c Accessed: 2022-05-22.
[3] Society, A. C. Scifindern700: A cas solution. Https://scifinder-n.cas.org. Accessed: 2022-05-22.
[4] Dai, H., Li, C., Coley, C., Dai, B. & Song, L. Retrosynthesis prediction with conditional graph logic network. In Wallach, H. et al. (eds.) Advances in Neural Information Processing Systems, vol. 32, 8872-8882 (Curran Associates, Inc., 2019).
[5] Shi, C., Xu, M., Guo, H., Zhang, M. & Tang, J. A graph to graphs framework for retrosynthesis prediction. In III, H. D. & Singh, A. (eds.) Proceedings of the 37th International Conference on Machine Learning, vol. 119 of Proceedings of Machine Learning Research, 8818-8827 (PMLR, 2020).
[6] Sun, R., Dai, H., Li, L., Kearnes, S. & Dai, B. Towards understanding retrosynthesis by energy-based models. In Ranzato, M., Beygelzimer, A., Dauphin, Y., Liang, P. & Vaughan, J. W. (eds.) Advances in Neural Information Processing Systems, vol. 34, 10186-10194 (Curran Associates, Inc., 2021).
[7] Somnath, V. R., Bunne, C., Coley, C. W., Krause, A. & Barzilay, R. Learning graph models for retrosynthesis prediction. In Beygelzimer, A., Dauphin, Y., Liang, P. & Vaughan, J. W. (eds.) Advances in Neural Information Processing Systems (2021).
[8] Vaswani, A. et al. Attention is all you need. 710 In Guyon, I. et al. (eds.) Advances in Neural Information Processing Systems, vol. 30 (Curran Associates, Inc., 2017).
[9] Kipf, T. N. & Welling, M. Semi-supervised classification with graph convolutional networks. In 5th International Conference on Learning Representations, ICLR 2017, Toulon, France, Apr. 24-26, 2017, Conference Track Proceedings (OpenReview.net, 2017). URL https://openreview.net/forum?id=SJU4ayYgl.
[10] Kingma, D. P. & Welling, M. Auto-encoding variational bayes. In Bengio, Y. & LeCun, Y. (eds.) 2nd International Conference on Learning Representations, ICLR 2014, Banff, AB, Canada, Apr. 14-16, 2014, Conference Track Proceedings (2014). URL http://arxiv.org/abs/1312.6114.
[11] Segler, M. H. S., Preuss, M. & Waller, M. P. Planning chemical syntheses with deep neural networks and symbolic Al. Nature 555, 604{610 (2018).
[12] Yan, C. et al. Retroxpert: Decompose retrosynthesis prediction like a chemist. In Larochelle, H., Ranzato, M., Hadsell, R., Balcan, M. F. & Lin, H. (eds.) Advances in Neural Information Processing Systems, vol. 33, 11248{11258 (Curran Associates, Inc., 2020).
[13] Zheng, S., Rao, J., Zhang, Z., Xu, J. & Yang, Y. Predicting retrosynthetic reactions using self-corrected transformer neural networks. Journal of Chemical Information and Modeling 60, 47{55 (2019).
[14] Chen, B., Shen, T., Jaakkola, T. S. & Barzilay, R. Learning to make generalizable and diverse predictions for retrosynthesis 1910.09688v1.
[15] Kim, E., Lee, D., Kwon, Y., Park, M. S. & Choi, Y.-S. Valid, plausible, and diverse retrosynthesis using tied two-way transformers with latent variables. Journal of Chemical Information and Modeling 61, 123-133 (2021).
[16] Szymkuc, S. et al. Computer-assisted synthetic planning: The end of the beginning. Angewandte Chemie International Edition 55, 5904-5937 (2016).
[17] Coley, C. W., Rogers, L., Green, W. H. & Jensen, K. F. Computer-assisted retrosynthesis based on molecular similarity. ACS Central Science 3, 1237-1245 (2017).
[18] Segler, M. H. S. & Waller, M. P. Neural-symbolic machine learning for retrosynthesis and reaction prediction. Chemistry—A European Journal 23, 5966-5971 (2017).
[19] Seidl, P. et al. Improving few- and zero-shot reaction template prediction using modern hopfield networks. Journal of Chemical Information and Modeling 62, 2111-2120 (2022).
[20] Chen, S. & Jung, Y. Deep retrosynthetic reaction prediction using local reactivity and global attention. JACS Au 1, 1612-1620 (2021).
[21] Mao, K. et al. Molecular graph enhanced transformer for retrosynthesis prediction. Neurocomputing 457, 193{202 (2021).
[22] Irwin, R., Dimitriadis, S., He, J. & Bjerrum, E. J. Chemformer: a pre-trained transformer for computational chemistry. Machine Learning: Science and Technology 3, 015022 (2022).
[23] Tu, Z. & Coley, C. W. Permutation invariant graph-to-sequence model for template-free retrosynthesis and reaction prediction J. Chem. Inf. Model. 62, 3503-3513 (2022).
[24] Tetko, I. V., Karpov, P., Deursen, R. V. & Godin, G. State-of-the-art augmented NLP transformer models for direct and single-step retrosynthesis. Nat. Commun. 11, 5575 (2020).
[25] Wan, Y., Liao, B., Hsieh, C.-Y. & Zhang, S. Retroformer: Pushing the limits of interpretable end-to-end retrosynthesis transformer. In Chaudhuri, K. et al. (eds.) Proceedings of the 39th International Conference on Machine Learning, vol. 162, 22475-22490 (PMLR, 2022).
[26] Wang, X. et al. RetroPrime: A diverse, plausible and transformer-based method for single-step retrosynthesis predictions. Chemical Engineering Journal 420, 129845 (2021).
[27] Sacha, M. et al. Molecule edit graph attention network: Modeling chemical reactions as sequences of graph edits. Journal of Chemical Information and Modeling 61, 3273-3284 (2021).
[28] Corey, E. J. General methods for the construction of complex molecules. Pure and Applied Chemistry 14, 19-38 (1967).
[29] Lowe, D. M. Extraction of chemical structures and reactions from the literature (2012).
[30] Schneider, N., Stieff, N. & Landrum, G. A. What's what: The (nearly) definitive guide to reaction role assignment. Journal of Chemical Information and Modeling 56, 2336-2346 (2016).
[31] Seo, S.-W. et al. GTA: Graph truncated attention for retrosynthesis. Proceedings of the AAAI Conference on Artificial Intelligence 35, 531-539 (2021).
[32] Brown, D. G. & Bostrom, J. Analysis of past and present synthetic methodologies on medicinal chemistry: Where have all the new reactions gone?Journal of Medicinal Chemistry 59, 4443-4458 (2015).
[33] Vijayakumar, A. K. et al. Diverse beam search: Decoding diverse solutions from neural sequence models 1610.02424v2.
[34] Miyaura, N. & Suzuki, A. Palladium-catalyzed cross-coupling reactions of organoboron compounds. Chemical Reviews 95, 2457-2483 (1995).
[35] LeBlond, C. R., Andrews, A. T., Sun, Y. & Sowa, J. R. Activation of aryl chlorides for suzuki cross-coupling by ligandless, heterogeneous palladium. Organic Letters 3, 1555-1557 (2001).
[36] Yin & Liebscher, J. Carbon-carbon coupling reactions catalyzed by heterogeneous palladium catalysts. Chemical Reviews 107, 133-173 (2006).
[37] Fanta, P. E. The ullmann synthesis of biaryls. Synthesis 1974, 9-21 (1974).
[38] Stille, J. K. The palladium-catalyzed cross-coupling reactions of organotin reagents with organic electrophiles[new synthetic methods(58)]. Angewandte Chemie International Edition in English 25, 508-524 (1986).
[39] Tamao, K., Sumitani, K. & Kumada, M. Selective carbon-carbon bond formation by cross-coupling of grignard reagents with organic halides. catalysis by nickel-phosphine complexes. Journal of the American Chemical Society 94, 4374-4376 (1972).
[40] Yan, C. et al. Retroxpert. https://github.com/uta-smile/RetroXpert (2021).
[41] Somnath, V. R. (2021). Https://github.com/uta-smile/RetroXpert/issues/15#issuecomment-864845942 Accessed: 2022-06-01.
[42] Jin, W., Coley, C., Barzilay, R. & Jaakkola, T. Predicting organic reaction outcomes with weisfeiler-lehman network. In Guyon, I. et al. (eds.) Advances in Neural Information Processing Systems, vol. 30 (Curran Associates, Inc., 2017).
[43] Bradshaw, J., Kusner, M. J., Paige, B., Segler, M. H. S. & Hernandez-Lobato, J. M. A generative model for electron paths. In International Conference on Learning Representations (2019).
[44] Chen, Z., Min, M. R., Parthasarathy, S. & Ning, X. A deep generative model for molecule optimization via one fragment modification. Nature Machine Intelligence 3, 1040-1049 (2021).
[45] Jin, W., Barzilay, D. & Jaakkola, T. Hierarchical generation of molecular graphs using structural motifs. In III, H. D. & Singh, A. (eds.) Proceedings of the 37th International Conference on Machine Learning, vol. 119 of Proceedings of Machine Learning Research, 4839-4848 (PMLR, 2020).
[46] Degen, J., Wegscheid-Gerlach, C., Zaliani, A. & Rarey, M. On the art of compiling and using ‘drug-like’ chemical fragment spaces. ChemMedChem 3, 1503-1507 (2008).
[47] Ertl, P., Schuffenhauer, A. Estimation of synthetic accessibility score of drug-like molecules based on molecular complexity and fragment contributions. J Cheminform 1, 8 (2009). http://doi.org/10.1186/1758-2946-1-8.
[48] von Korff, M., Sander, T. Molecular Complexity Calculated by Fractal Dimension. Sci Rep9, 967 (2019). https://doi.org/10.1038/s41598-018-37253-8.
[49] Jin, W., Coley, C., Barzilay, R. & Jaakkola, T. Predicting Organic Reaction Outcomes with Weisfeiler-Lehman Network. in Advances in Neural Information Processing Systems (eds. Guyon, I. et al.) 30, (Curran Associates, Inc., 2017).
[50] Schwaller, P. et al. Molecular Transformer: A Model for Uncertainty-Calibrated Chemical Reaction Prediction. {ACS}Cent. Sci. 5, 1572-1583 (2019).
[51] Bi, H. et al. Non-Autoregressive Electron Redistribution Modeling for Reaction Prediction. in Proceedings of the 38th International Conference on Machine Learning (eds. Meila, M. & Zhang, T.) 139, 904-913 (PMLR, 2021).
[52] Do, K., Tran, T. & Venkatesh, S. Graph Transformation Policy Network for Chemical Reaction Prediction. in Proceedings of the 25th {ACM} {SIGKDD} International Conference on Knowledge Discovery & Data Mining (ACM, 2019). doi:10.1145/3292500.3330958.
[53] Qian, W. W. et al. Integrating Deep Neural Networks and Symbolic Inference for Organic Reactivity Prediction. (2020). doi:10.26434/chemrxiv.11659563.v1.
[54] Chen, B., Li, C., Dai, H. & Song, L. Retro*: Learning retrosynthetic planning with neural guided A* search. In III, H. D. & Singh, A. (eds.) Proceedings of the 37th International Conference on Machine Learning, vol. 119, 1608-1616 (PMLR, 2020).
[55] Blakemore, D. C. et al. Organic synthesis provides opportunities to transform drug discovery. Nat. Chem. 10, 383-394 (2018).
[56] Lajiness, M. S., Maggiora, G. M. & Shanmugasundaram, V. Assessment of the consistency of medicinal chemists in reviewing sets of compounds. J. Med. Chem 47, 4891-4896 (2004).
[57] Huang, Q., Li, L.-L. & Yang, S.-Y. RASA: A rapid retrosynthesis-based scoring method for the assessment of synthetic accessibility of drug-like molecules. J. Chem. Inf. Model. 51, 2768-2777 (2011).
[58] Takaoka, Y. et al. Development of a method for evaluating drug-likeness and ease of synthesis using a data set in which compounds are assigned scores based on chemists' intuition. J. Chem. Inf. Comput. Sci. 43, 1269-1275 (2003).
[59] Kutchukian, P. S. et al. Inside the mind of a medicinal chemist: The role of human bias in compound prioritization during drug discovery. PLoS ONE 7, e48476 (2012).
[60] Gabrielson, S. W. SciFinder. J. Med. Libr. Assoc. 106 (2018).
[61] Kearnes, S. M. et al. The open reaction database. J. Am. Chem. Soc. 143, 18820-18826 (2021).
[62] Lowe, D. M. Chemical reactions from us patents (1976-sep2016). https://doi.org/10.6084/m9.gshare.5104873.v1 Accessed: 2022-11-06.
[63] Chen, F., Wang, Y.-C., Wang, B. & Kuo, C.-C. J. Graph representation learning: a survey. APSIPA Transactions on Signal and Information Processing 9 (2020).
[64] Murray, C. & Rees, D. The rise of fragment-based drug discovery. Nat. Chem. 1, 187-192 (2009).
[65] Hajduk, P. J. & Greer, J. A decade of fragment-based drug design: strategic advances and lessons learned. Nat. Rev. Drug Discov. 6, 211-219 (2007).
[66] Jin, W., Barzilay, R. & Jaakkola, T. Junction tree variational autoencoder for molecular graph generation. In Dy, J. & Krause, A. (eds.) Proceedings of the 35th International Conference on Machine Learning, vol. 80, 2323-2332 (PMLR, 2018).
[67] Podda, M., Bacciu, D. & Micheli, A. A deep generative model for fragment-based molecule generation. In Chiappa, S. & Calandra, R. (eds.) Proceedings of the Twenty Third International Conference on Artificial Intelligence and Statistics, vol. 108, 2240-2250 (PMLR, 2020).
[68] Zhang, Z., Liu, Q., Wang, H., Lu, C. & Lee, C.-K. Motif-based graph self-supervised learning for molecular property prediction. In Ranzato, M., Beygelzimer, A., Dauphin, Y., Liang, P. & Vaughan, J. W. (eds.) Advances in Neural Information Processing Systems, vol. 34, 15870-15882 (Curran Associates, Inc., 2021).
[69] Lewis, M. et al. BART: Denoising sequence-to-sequence pre-training for natural language generation, translation, and comprehension. In Proceedings of the 58th Annual Meeting of the Association for Computational Linguistics (2020).
[70] DimGorr. http://github.com/DeepGraphLearning/torchdrug/issues/131 Accessed: 2022-10-01.
[71] Lin, Y. et al. Reinforcing the supply chain of umifenovir and other antiviral drugs with retrosynthetic software. Nat. Commun. 12 (2021).
[72] Mikulak-Klucznik, B. et al. Computational planning of the synthesis of complex natural products. Nature 588, 83-88 (2020).
[73] Keam, S. J. Mavacamten: First approval. Drugs 82, 1127-1135 (2022).
[74] Oslob, J. et al. Pyrimidinedione compounds. US20170281626A1 (2017).
[75] Al-Samkari, H. & van Beers, E. J. Mitapivat, a novel pyruvate kinase activator, for the treatment of hereditary hemolytic anemias. Therapeutic Advances in Hematology 12, 204062072110660 (2021).
[76] Sizemore, J. P., Guo, L., Mirmehrabi, M. & Su, Y. WO2019104134A1 (2019).
[77] Hoy, S. M. Oteseconazole: First approval. Drugs 82, 1017{1023 (2022).
[78] Wirth, D. D., Yates, C. M. & Hoekstra, W. J. WO2017049096A1 (2017).
[79] Keam, S. J. Tapinarof cream 1%: First approval. Drugs 82, 1221-1228 (2022).
[80] Chen, G., Webster, J., Li, J., Hu, K. & Zhu, J. WO2001042231A2 (2001).
[81] Duan, X.-F., Zeng, J., Lu, J.-W. & Zhang, Z.-B. Insights into the general and efficient cross McMurry reactions between ketones. The Journal of Organic Chemistry 71, 9873-9876 (2006).
[82] Robiette, R., Richardson, J., Aggarwal, V. K. & Harvey, J. N. Reactivity and selectivity in the witting reaction: a computational study. J. Am. Chem. Soc. 128, 2394-2409 (2006).
[83] Yan, C. et al. Retroxpert: Decompose retrosynthesis prediction like a chemist. In Larochelle, H., Ranzato, M., Hadsell, R., Balcan, M. F. & Lin, H. (eds.) Advances in Neural Information Processing Systems, vol. 33, 11248-11258 (Curran Associates, Inc., 2020).
[84] Gaussian 16, Revision C.01, Frisch, M. J.; Trucks, G. W.; Schlegel, H. B.; Scuseria, G. E.; Robb, M. A.; Cheeseman, J. R.; Scalmani, G.; Barone, V.; Petersson, G. A.; Nakatsuji, H.; Li, X.; Caricato, M.; Marenich, A. V.; Bloino, J.; Janesko, B. G.; Gomperts, R.; Mennucci, B.; Hratchian, H. P.; Ortiz, J. V.; Izmaylov, A. F.; Sonnenberg, J. L.; Williams-Young, D.; Ding, F.; Lipparini, F.; Egidi, F.; Goings, J.; Peng, B.; Petrone, A.; Henderson, T.; Ranasinghe, D.; Zakrzewski, V. G.; Gao, J.; Rega, N.; Zheng, G.; Liang, W.; Hada, M.; Ehara, M.; Toyota, K.; Fukuda, R.; Hasegawa, J.; Ishida, M.; Nakajima, T.; Honda, Y.; Kitao, O.; Nakai, H.; Vreven, T.; Throssell, K.; Montgomery, J. A., Jr.; Peralta, J. E.; Ogliaro, F.; Bearpark, M. J.; Heyd, J. J.; Brothers, E. N.; Kudin, K. N.; Staroverov, V. N.; Keith, T. A.; Kobayashi, R.; Normand, J.; Raghavachari, K.; Rendell, A. P.; Burant, J. C.; Iyengar, S. S.; Tomasi, J.; Cossi, M.; Millam, J. M.; Klene, M.; Adamo, C.; Cammi, R.; Ochterski, J. W.; Martin, R. L.; Morokuma, K.; Farkas, O.; Foresman, J. B.; Fox, D. J. Gaussian, Inc., Wallingford CT, 2016.
[85] D. A. Case, H. M. Aktulga, K. Belfon, I. Y. Ben-Shalom, J. T. Berryman, S. R. Brozell, D. S. Cerutti, T. E. Cheatham, III, G. A. Cisneros, V. W. D. Cruzeiro, T. A. Darden, R. E. Duke, G. Giambasu, M. K. Gilson, H. Gohlke, A. W. Goetz, R. Harris, S. Izadi, S. A. Izmailov, K. Kasavajhala, M. C. Kaymak, E. King, A. Kovalenko, T. Kurtzman, T. S. Lee, S. LeGrand, P. Li, C. Lin, J. Liu, T. Luchko, R. Luo, M. Machado, V. Man, M. Manathunga, K. M. Merz, Y. Miao, O. Mikhailovskii, G. Monard, H. Nguyen, K. A. O'Hearn, A. Onufriev, F. Pan, S. Pantano, R. Qi, A. Rahnamoun, D. R. Roe, A. Roitberg, C. Sagui, S. Schott-Verdugo, A. Shajan, J. Shen, C. L. Simmerling, N. R. Skrynnikov, J. Smith, J. Swails, R. C. Walker, J. Wang, J. Wang, H. Wei, R. M. Wolf, X. Wu, Y. Xiong, Y. Xue, D. M. York, S. Zhao, and P. A. Kollman (2022), Amber 2022, University of California, San Francisco.

1. A method of performing retrosynthesis to predict output reactants molecules that can be used in a synthetic molecule model for an input target molecule, the method comprising:
receiving, by a processor, an input molecule data structure represented as a graph data object;
evaluating, by the processor, one or more graph data objects of the input molecule data structure, wherein evaluating includes a set of sequences to:
determine, by the processor, via one or more trained AI algorithm via a training data set of molecule data structures, for one or more reaction center types, a likelihood value that a given bond of a plurality of assessed bonds or a given atom of a plurality of assessed atoms of each of one or more graph data objects is likely a reaction center for a subsequent synthesis, wherein the one or more reaction center types include:
i) a first reaction center type associated with a new chemical bond formation that is formed during a hypothetical reaction and exists in the input target molecule but not in the output reactants molecules;
ii) a second reaction center type associated with an existing chemical bond in the input molecule data structure being changed in type during a hypothetical reaction; and/or
iii) a third reaction center type associated with an atom in the input molecule data structure connected with fragments being removed during a hypothetical reaction without a new chemical bond being formed or a type of the existing chemical bond being changed,
wherein the likelihood values of the plurality of assessed bonds of each of one or more graph data objects are used in a selection of one or more reaction centers for the one or more graph data objects to generate a plurality of intermediate molecular graph data objects each having a modified chemical bond at the selected one or more reaction centers for the one or more graph data objects, wherein one or more of the plurality of intermediate molecular graph data objects with the modified chemical bond are attached to a chemical fragment at the modified chemical bond to generate a completed reactant graph, wherein the completed reactant graph is used to generate the synthetic molecule model.

2. The method of claim 1, wherein the one or more of the plurality of intermediate molecular graph data objects with the modified chemical bond are generated by:
determining, by the processor, a likelihood of a neighbor bond type change for a given reaction center.

3. The method of claim 2, wherein the likelihood of the neighbor bond type change for the given reaction center is determined using a second trained AI algorithm that learns for a given bond structure and on a structure of the graph data objects of the training data set of molecule data structures.

4. The method of claim 1, wherein the one or more of the plurality of intermediate molecular graph data objects with the modified chemical bond are generated by:
determining, by the processor, a likelihood of an atom charge change for all the atoms involved in a given reaction center and their associated neighboring bonds.

5. The method of claim 4, wherein the likelihood of the atom charge change is determined using a third trained AI algorithm that learns, via training data set of molecule data structures, for a given atom structure probabilities of accepting one electron, donating one electron, or no electron change during a hypothetical reaction

6. The method of claim 1, wherein the one or more of the plurality of intermediate molecular graph data objects with the modified chemical bond are generated by:
iteratively attaching, by the processor, a chemical substructure to a given intermediate molecular graph data object of the plurality of intermediate molecular graph data objects based on a fourth trained AI algorithm that learns for a given atom structure and on a structure of the graph data objects of the training data set of molecule data structures.

7. The method of claim 6, wherein iteratively attaching the chemical substructure includes:
determining, by the processor, likelihood values for inclusion of one or more types of new substructure from a library of candidate substructures each comprising a bond or a ring structure, wherein the likelihood values are determined using a fifth trained AI algorithm that learns for a given atom structure and on a structure of the graph data objects of the training data set of molecule data structures.

8. The method of claim 1, wherein the one or more graph data objects include a molecular graph and a BRICS graph.

9. The method of claim 1, wherein the one or more graph data objects include a molecular graph and a junction tree.

10. The method of claim 1, wherein the selection of one or more reactions for the one or more graph data objects includes:
generating, by the processor, a ranked list of candidate reactant graphs; and
selecting, by the processor, via a beam search, a pre-defined number of top reaction centers as the one or more reaction centers for the one or more graph data objects to generate the plurality of intermediate molecular graph data objects.

11. The method of claim 1, wherein the likelihood value that the given bond of the plurality of assessed bonds of each of one or more graph data objects is likely the first reaction center type is determined based on a sixth trained Al algorithm, wherein the sixth trained AI algorithm operation is configured to learn, using the training data set of molecule data structures, based on a given bond structure and on a structure of the graph data objects.

12. The method of claim 1, wherein the likelihood value that the given bond of the plurality of assessed bonds of each of one or more graph data objects is likely the second reaction center type is determined based on a seventh trained AI algorithm, wherein the seventh trained AI algorithm is configured to learn, using the training data set of molecule data structures, based on a given bond structure and on a structure of the graph data objects.

13. The method of claim 1, wherein the likelihood value that the given atom of the plurality of assessed atoms of each of one or more graph data objects is likely the third reaction center type is determined based on an eighth trained Al algorithm, wherein the eighth trained AI algorithm is configured to learn, using the training data set of molecule data structures, based on an atom and a structure of the graph data objects.

14. The method of claim 1, wherein the input molecule data structure is evaluated along with a plurality of input molecule data structures in a pipeline operation.

15. The method of claim 1, wherein the plurality of input molecule data structures has an average molecule size between 20-500 atoms or rings.

16. The method of claim 1, wherein the input molecule data structure is of an existing therapeutic molecule or a pharmaceutically active molecule.

17. The method of claim 6, wherein the chemical substructure is iteratively attached in a ranked manner from the library of candidate substructures without any chemical rules or templates imposed for the attachment.

18. The method of claim 1, wherein the input molecule data structure was generated by molecule optimization evaluation for drug discovery analysis.

19. (canceled)

20. (canceled)

21. A system, comprising:
a processor; and
a memory operatively connected to the processor, the memory having instructions stored thereon that, when executed by the processor, cause the processor to:
receive an input molecule data structure represented as a graph data object;
evaluate one or more graph data objects of the input molecule data structure, wherein evaluating includes a set of sequences to:
determine via one or more trained AI algorithm via a training data set of molecule data structures, for one or more reaction center types, a likelihood value that a given bond of a plurality of assessed bonds or a given atom of a plurality of assessed atoms of each of one or more graph data objects is likely a reaction center for a subsequent synthesis, wherein the one or more reaction center types include:
i) a first reaction center type associated with a new chemical bond formation that is formed during a hypothetical reaction and exists in an input target molecule but not in an output reactants molecules;
ii) a second reaction center type associated with an existing chemical bond in the input molecule data structure being changed in type during a hypothetical reaction; and/or
iii) a third reaction center type associated with an atom in the input molecule data structure connected with fragments being removed during a hypothetical reaction without a new chemical bond being formed or a type of the existing chemical bond being changed,
wherein the likelihood values of the plurality of assessed bonds of each of one or more graph data objects are used in a selection of one or more reaction centers for the one or more graph data objects to generate a plurality of intermediate molecular graph data objects each having a modified chemical bond at the selected one or more reaction centers for the one or more graph data objects, wherein one or more of the plurality of intermediate molecular graph data objects with the modified chemical bond are attached to a chemical fragment at the modified chemical bond to generate a completed reactant graph, wherein the completed reactant graph is used to generate a synthetic molecule model.

22. A non-transitory computer-readable storage medium having instructions stored thereon that, when executed by a processor, cause the processor to:
receive an input molecule data structure represented as a graph data object;
evaluate one or more graph data objects of the input molecule data structure, wherein evaluating includes a set of sequences to:
determine via one or more trained AI algorithm via a training data set of molecule data structures, for one or more reaction center types, a likelihood value that a given bond of a plurality of assessed bonds or a given atom of a plurality of assessed atoms of each of one or more graph data objects is likely a reaction center for a subsequent synthesis, wherein the one or more reaction center types include:
i) a first reaction center type associated with a new chemical bond formation that is formed during a hypothetical reaction and exists in an input target molecule but not in an output reactants molecules;
ii) a second reaction center type associated with an existing chemical bond in the input molecule data structure being changed in type during a hypothetical reaction; and/or
iii) a third reaction center type associated with an atom in the input molecule data structure connected with fragments being removed during a hypothetical reaction without a new chemical bond being formed or a type of the existing chemical bond being changed,
wherein the likelihood values of the plurality of assessed bonds of each of one or more graph data objects are used in a selection of one or more reaction centers for the one or more graph data objects to generate a plurality of intermediate molecular graph data objects each having a modified chemical bond at the selected one or more reaction centers for the one or more graph data objects, wherein one or more of the plurality of intermediate molecular graph data objects with the modified chemical bond are attached to a chemical fragment at the modified chemical bond to generate a completed reactant graph, wherein the completed reactant graph is used to generate a synthetic molecule model.
